(19) United States
(12) Patent Application Publication
(10) Pub. No.: US 2025/0319297 A1
(43) Pub. Date: Oct. 16, 2025

(54) DISINFECTING CAP FOR FLUID PATH ELEMENT

(71) Applicant: BAYER HEALTHCARE LLC, WHIPPANY, NJ (US)

(72) Inventors: MICHAEL SPOHN, FENELTON, PA (US); KEVIN COWAN, ALLISON PARK, PA (US); JOHN HAURY, SEWICKLEY, PA (US); ROBERT BAIKO, PITTSBURGH, PA (US); COREY SAVANNAH, BUTLER, PA (US); THEODORE MOSLER, RALEIGH, NC (US); JOSEPH RANALLETTA, GREENVILLE, SC (US); MATTHEW WALKER, LILLINGTON, NC (US); SAYED PEERZADE, FUQUAY VARINA, NC (US); JAMES FENTRESS, CREEDMOOR, NC (US)

(21) Appl. No.: 18/868,905

(22) PCT Filed: Jun. 13, 2023
(86) PCT No.: PCT/US2023/025159

§ 371 Date: Nov. 25, 2024

Related U.S. Application Data
(60) Provisional application No. 63/351,882, filed on Jun. 14, 2022.
(60) Provisional application No. 63/409,300, filed on Sep. 23, 2022.

(51) Int. Cl. A61M 39/16; A61M 5/00; A61M 39/10

(52) U.S. Cl. CPC A61M 39/162; A61M 5/007; A61M 39/105; A61M 2205/581; A61M 2205/582

(57) ABSTRACT

Provided is a disinfecting cap for a fluid path element having an inner lumen and an outer cylindrical wall surrounding the inner lumen. The disinfecting cap includes a housing configured to receive the inner lumen and the outer cylindrical wall of the fluid path element. The housing has an open proximal end, a closed distal end, and a sidewall extending between the open proximal end and the closed distal end to define an interior volume. A sleeve protrudes proximally from an inner surface of the closed distal end and defines an opening configured to receive the inner lumen of the fluid path element. A compressible absorbent material at least partially saturated with a disinfecting fluid is disposed within the interior volume of the housing and surrounding at least a portion of the sleeve.

### CROSS-REFERENCE TO RELATED APPLICATIONS

This application claims priority to U.S. Provisional Application No. 63/351,882, filed on Jun. 14, 2022, and U.S. Provisional Application No. 63/409,300, filed on Sep. 23, 2022, the disclosures of which are incorporated herein by reference in their entirety.

### BACKGROUND OF THE DISCLOSURE

### Field of the Disclosure

The present disclosure is related generally to features associated with the multi-patient fluid path elements for powered medical fluid injection systems and, especially, to caps for fluid path elements that prevent contamination with microbial contaminants before and between serial fluid injection procedures which utilize a common multi-patent fluid path set.

### Background

Syringe injection systems are among the medical devices used in medical imaging procedures. To reduce the number of disposable components during a sequence of fluid injections, systems may incorporate a multi-patient portion, such as a multi-patient pump system and multi-patient fluid path elements, that may be used over a series of fluid injection procedures; and a single-patient fluid path element, including associated check valves, that are used for only a single injection procedure and then disposed of and replace with a new, sterile single-patient fluid path element for a subsequent fluid injection procedure.

A number of syringe-based or peristaltic pump-based powered injectors have been developed for use in medical procedures such as cardiovascular angiography (CV), computed tomography (CT) and nuclear magnetic resonance (NMR)/magnetic resonance imaging (MRI). Some of these powered injectors may include options for use in a multi-patient configuration utilizing multi-patient components and single-patient components. One example of such a system is the Bayer MEDRAD® Centargo CT Fluid Injection System, including multi-patient and single-patient components as described in U.S. Pat. Nos. 10,507,319 and 10,549,084, respectively, the disclosures of which are incorporated by reference herein. Another example of such a system is the Bayer AVANTA® Fluid Injection System.

Since the various multi-patient elements are used over several fluid injection procedures, steps must be taken or the element design must incorporate features to ensure that these elements are not exposed to or are sterilized to remove microbial contaminants that may be harmful to later patients before the next injection procedure. For example, many systems may include a swabable valve that may be disinfected by a technician wiping the valve with an alcohol-soaked wipe in between injection procedures.

Conventional systems having swabable valves include standard Luer-type connectors between the multi-patient and single-patient components which are fitted with off-the-shelf swabable valves on the female Luer component and may include a threaded connection mechanism. Examples of a threaded connector for use in a multi-patient configuration are described in U.S. Pat. No. 8,540,698, the disclosure of which is incorporated by reference herein. When performing multiple fluid injection procedures in a high throughput situation, connecting, and disconnecting threaded connectors and manually wiping swabable valves is time consuming and may reduce the efficiency of an injection suite. Further, such threaded connections may be susceptible to over tightening or under-tightening resulting in potential leaks or defecting connections.

Active disinfection when replacing used single patient fluid path elements between injection procedures may become more desired to ensure no contamination of the surfaces of the multi-patient fluid path elements. New systems for actively disinfecting multi-patient fluid path components in an efficient and effective manner, including simple connection methods are needed.

### SUMMARY

The present disclosure provides a disinfecting fluid component cap for use in ensuring a sterile connection between two fluid path components of a powered fluid injections in contrast enhanced imaging procedures, such as computed tomography (CT), angiography (CV), and magnetic resonance imaging (MRI) imaging procedures.

In some embodiments, provided is a disinfecting cap for a fluid path element. The disinfecting cap may include a housing configured to fit over at least a portion of the fluid path element. The housing may include an open proximal end, a closed distal end, and a sidewall extending between the open proximal end and the closed distal end to define an interior volume. The disinfecting cap further may include a compressible absorbent material at least partially saturated with a disinfecting fluid, and positioned within the interior volume of the housing. The disinfecting cap further may include an insert within the interior volume connected to the compressible absorbent material, the insert movable toward the closed distal end to compress the compressible absorbent material with movement of the fluid path element toward the closed distal end. The insert may include a fluid path sealing portion configured to seal a lumen on the fluid path element, and a circumferential flange extending around the fluid path sealing portion, the circumferential flange comprising one or more passageways configured to permit a flow of the disinfecting fluid from the compressible absorbent material to the fluid path element when the insert is urged toward the closed distal end by the fluid path element.

In some embodiments, an inner surface of the sidewall may include one or more radially inwardly protruding projections configured to removably engage the fluid path element and retain the disinfecting cap on the fluid path element. Movement of the insert toward the closed distal end via movement of the fluid path element may compress the compressible absorbent material and release at least a portion of the disinfecting fluid through the one or more passageways so that the disinfecting fluid contacts at least a portion of the fluid path element.

In some embodiments, the circumferential flange may be attached to an inner surface of the sidewall of the housing. The fluid path sealing portion may include a rubber material, a pliable plastic material, or a silicone material configured to create a fluid tight seal with the lumen of the fluid path element. The absorbent material may be a sponge or cotton. The disinfecting fluid may include isopropyl alcohol, ethanol, a combination thereof, or an aqueous solution thereof.

In some embodiments, a gripping flange may protrude distally from the closed distal end. A seal may be removably connected to the open proximal end, wherein the seal fluidly seals the open proximal end. The seal may include a pull tab protruding radially outward relative to the sidewall of the housing. The pull tab may be configured to remove the seal from the open proximal end.

In some embodiments, a second compressible absorbent material may be provided on a proximal surface of the insert and surrounding the fluid path sealing portion. The insert may be threadably connected to the housing such that rotation of the insert relative to the housing moves the insert toward the closed distal end to compress the compressible absorbent material and release at least a portion of the disinfecting fluid.

In some embodiments, the fluid path sealing portion may include a key configured to engage with the fluid path element to rotate the insert relative to the housing with rotation of the disinfecting cap. An inner surface of the closed distal end may include one or more ribs configured to prevent rotation of the compressible absorbent material relative to the housing. A proximal end of the compressible absorbent material may include a groove configured to receive at least a portion of the sidewall of the fluid path element.

In some embodiments, a disinfecting cap for a fluid path element may include a housing configured to fit over at least a portion of the fluid path element, the housing having an open proximal end, an open distal end, and a sidewall extending between the open proximal end and the open distal end. The disinfecting cap further may include a flange extending across an interior of the housing between the open proximal end and the open distal end, the flange having one or more openings. The disinfecting cap further may include a compressible absorbent material at least partially saturated with a disinfecting fluid, wherein the compressible absorbent material may be at a distal end of the flange. The disinfecting cap further may include a plunger connected to the housing to enclose the open distal end, the plunger being slidably movable relative to the housing between the open distal end and the open proximal end. Movement of the plunger in a proximal direction may compress the compressible absorbent material and release at least a portion of the disinfecting fluid through the one or more openings.

In some embodiments, the flange may include a sealing surface configured to seal a lumen on the fluid path element. A second compressible absorbent material may be provided on a proximal end of the flange and surrounding the sealing surface.

In some embodiments, the plunger may include a collapsible fluid bulb containing the disinfecting fluid, wherein the collapsible fluid bulb is collapsible with a pushing movement in a proximal direction to dispense the disinfecting fluid into the housing through one or more holes in a proximal end of the plunger. An inner surface of the sidewall may include one or more radially inwardly protruding projections configured to removably engage the fluid path element and retain the disinfecting cap on the fluid path element.

In some embodiments, a disinfecting cap for a fluid path element having an inner lumen and an outer cylindrical wall surrounding the inner lumen may include a housing configured to receive the inner lumen and the outer cylindrical wall of the fluid path element. The housing may include an open proximal end, a closed distal end, and a sidewall extending between the open proximal end and the closed distal end to define an interior volume. The disinfecting cap further may include a sleeve protruding proximally from an inner surface of the closed distal end, the sleeve defining an opening configured to receive the inner lumen of the fluid path element. The disinfecting cap further may include a compressible absorbent material at least partially saturated with a disinfecting fluid, the compressible absorbent material disposed within the interior volume of the housing and surrounding at least a portion of an outer portion of the sleeve. The outer cylindrical wall of the fluid path element may be configured to be received in a disinfecting space between an inner surface of the housing and the sleeve such that movement of the fluid path element toward the closed distal end compresses the compressible absorbent material and releases at least a portion of the disinfecting fluid into the disinfecting space.

In some embodiments, an inner surface of the sleeve may be configured to be in sealing engagement with the inner lumen of the fluid path element. The compressible absorbent material may extend substantially from the closed distal end to the proximal end of the housing.

In some embodiments, the sleeve may include one or more longitudinal ribs protruding radially outward and configured to engage the compressible absorbent material surrounding the outer portion of the sleeve to prevent rotation of the compressible absorbent material relative to the sleeve. The compressible absorbent material may have an axial slot configured to receive the outer cylindrical wall of the fluid path element. The sleeve may include a tab configured to provide a tactile or audio feedback when the inner lumen of the fluid path element is fully inserted into the sleeve.

In some embodiments, provided is a fluid path assembly that may include a fluid path element having an inner lumen and an outer cylindrical wall surrounding the inner lumen; and a disinfecting cap configured to connect to the fluid path element. The disinfecting cap may include a housing configured to receive the inner lumen and the outer cylindrical wall of the fluid path element, the housing including an open proximal end, a closed distal end, and a sidewall extending between the open proximal end and the closed distal end to define an interior volume. The disinfecting cap further may include a sleeve protruding proximally from an inner surface of the closed distal end, the sleeve defining an opening configured to receive the inner lumen of the fluid path element. The disinfecting cap further may include a compressible absorbent material at least partially saturated with a disinfecting fluid, the compressible absorbent material disposed within the interior volume of the housing and surrounding at least a portion of an outer portion of the sleeve. The outer cylindrical wall of the fluid path element may be configured to be received in a disinfecting space between an inner surface of the housing and the sleeve such that movement of the fluid path element toward the closed distal end compresses the compressible absorbent material and releases at least a portion of the disinfecting fluid into the disinfecting space.

In some embodiments, the fluid path element may be a Luer connector, a multi-patient fluid path element, or a single-patient fluid path element. In some embodiments, the fluid path element may include a first connector element having a body, a first lumen, a first flexible leg, and a second flexible leg. The fluid path element further may include a second connector element having a body defining an undercut, a second lumen, a channel defined in the body, and at least one sealing element positioned within the channel. The first flexible leg may include a first flange and the second flexible leg comprises a second flange. Upon engagement of the first connector element with the second connector element, the first flange and the second flange may engage with the undercut of the body of the second connector element to prevent disengagement of the first connector element and the second connector element. The sealing element may be configured to define a fluid tight seal between the second lumen of the second connector element and the first lumen of the first connector element to form a fluid path when the first connector element and the second connector element are engaged with one another.

In addition to the illustrative aspects and features described above, further aspects and features will become apparent by reference to the drawings and the following detailed description.

### BRIEF DESCRIPTION OF THE DRAWINGS

The features of the various embodiments of the disinfecting caps described herein are set forth with particularity in the appended claims. Such features, however, both as to organization and methods of operation may be better understood by reference to the following description, taken in conjunction with the accompanying drawings.

FIG. 1A is a perspective view of a fluid path set for use with a fluid injector in accordance with one embodiment of the present disclosure;

FIG. 1B is a perspective view of a fluid path element of a fluid path set configured for connecting to a disinfecting cap described herein with various embodiments of the present disclosure;

FIG. 2A is a perspective view of a disinfecting cap for use with a fluid path element of a fluid path set in accordance with one embodiment of the present disclosure;

FIG. 2B is a side cross-sectional view of the disinfecting cap shown in FIG. 2A;

FIG. 2C is a perspective cross-sectional view of the disinfecting cap shown in FIG. 2A;

FIG. 2D is a perspective cross-sectional view of the disinfecting cap shown in FIG. 2A with a fluid path element shown connected to the disinfecting cap in a first position;

FIG. 2E is a perspective cross-sectional view of the disinfecting cap shown in FIG. 2A with a fluid path element shown connected to the disinfecting cap in a second position;

FIG. 2F is a perspective view of an insert of the disinfecting cap shown in FIG. 2B;

FIG. 3A is a perspective cross-sectional view of a disinfecting cap for use with a fluid path element of a fluid path set in accordance with another embodiment of the present disclosure;

FIG. 3B is a perspective cross-sectional view of the disinfecting cap shown in FIG. 3A with a fluid path element connected to the disinfecting cap;

FIG. 3C is a front perspective view of an insert of the disinfecting cap shown in FIG. 3A;

FIG. 3D is a rear perspective view of an insert of the disinfecting cap shown in FIG. 3A;

FIG. 4A is a perspective cross-sectional view of a disinfecting cap for use with a fluid path element of a fluid path set in accordance with another embodiment of the present disclosure;

FIG. 4B is a perspective cross-sectional view of the disinfecting cap shown in FIG. 4A with a fluid path element connected to the disinfecting cap;

FIG. 4C is a back perspective view of a compressible absorbent material on an insert of the disinfecting cap shown in FIG. 4A;

FIG. 5A is a perspective cross-sectional view of a disinfecting cap for use with a fluid path element of a fluid path set in accordance with another embodiment of the present disclosure;

FIG. 5B is a perspective cross-sectional view of the disinfecting cap shown in FIG. 5A with a fluid path element connected to the disinfecting cap;

FIG. 5C is a front perspective view of a compressible absorbent material of the disinfecting cap shown in FIG. 5A;

FIG. 6A is a perspective cross-sectional view of a disinfecting cap for use with a fluid path element of a fluid path set in accordance with another embodiment of the present disclosure;

FIG. 6B is a side cross-sectional view of the disinfecting cap shown in FIG. 6A with a fluid path element connected to the disinfecting cap;

FIG. 6C is a top view of a sliding member of the disinfecting cap shown in FIG. 6A;

FIG. 6D is a side perspective view of the sliding member of the disinfecting cap shown in FIG. 6A;

FIG. 7A is a perspective cross-sectional view of a disinfecting cap for use with a fluid path element of a fluid path set in accordance with another embodiment of the present disclosure;

FIG. 7B is a perspective cross-sectional view of the disinfecting cap shown in FIG. 7A with a fluid path element connected to the disinfecting cap;

FIG. 8A is a perspective cross-sectional view of a disinfecting cap for use with a fluid path element of a fluid path set in accordance with another embodiment of the present disclosure;

FIG. 8B is a perspective cross-sectional view of the disinfecting cap shown in FIG. 8A with a fluid path element connected to the disinfecting cap;

FIG. 9A is side cross-sectional view of a disinfecting cap for use with a fluid path element of a fluid path set in accordance with another embodiment of the present disclosure;

FIG. 9B is a side cross-sectional view of the disinfecting cap shown in FIG. 9A with a sealing member in an open state;

FIG. 10A is a side cross-sectional view of a disinfecting cap for use with a fluid path element of a fluid path set in accordance with another embodiment of the present disclosure;

FIG. 10B is a perspective view of a sleeve portion of a compressible absorbent material for use with the disinfecting cap shown in FIG. 10A;

FIG. 11A is a side cross-sectional view of a disinfecting cap for use with a fluid path element of a fluid path set in accordance with another embodiment of the present disclosure, with the disinfecting cap shown in a disconnected position;

FIG. 11B is a side cross-sectional view of the disinfecting cap shown in FIG. 11A with the disinfecting cap shown in a connected position;

FIG. 12A is a side cross-sectional view of a disinfecting cap for use with a fluid path element of a fluid path set in accordance with another embodiment of the present disclosure, with the disinfecting cap shown in a disconnected position;

FIG. 12B is a side cross-sectional view of the disinfecting cap shown in FIG. 12A with the disinfecting cap shown in a connected position;

FIG. 13A is a side cross-sectional view of a disinfecting cap for use with a fluid path element of a fluid path set in accordance with another embodiment of the present disclosure;

FIG. 13B is a side cross-sectional view of the disinfecting cap shown in FIG. 13A in combination with a fluid path element in a first position;

FIG. 13C is a side cross-sectional view of the disinfecting cap shown in FIG. 13A in combination with a fluid path element in a first position;

FIG. 14A is a perspective view of an outer housing of a disinfecting cap for use with a fluid path element of a fluid path set in accordance with another embodiment of the present disclosure;

FIG. 14B is a perspective view of an inner sealing member of the disinfecting cap;

FIG. 14C is a side cross-sectional view of an assembled disinfecting cap with a fluid path element connected to the disinfecting cap and the inner sealing member shown in a first position;

FIG. 14D is a side cross-sectional view of an assembled disinfecting cap with a fluid path element connected to the disinfecting cap and the inner sealing member shown in a second position;

FIG. 15A is a perspective cross-sectional view of a disinfecting cap for use with and connected to a fluid path element of a fluid path set in accordance with another embodiment of the present disclosure;

FIG. 15B is a perspective exploded view of the disinfecting cap and fluid path element shown in FIG. 15A;

FIG. 15C is a perspective view of an outer housing of the disinfecting cap shown in FIG. 15A;

FIG. 15D is a rear perspective view of a compressible absorbent material of the disinfecting cap shown in FIG. 15A;

FIG. 15E is a perspective view of an insertable tray of the disinfecting cap shown in FIG. 15A;

FIG. 16A is a perspective cross-sectional view of a disinfecting cap for use with a fluid path element of a fluid path set in accordance with another embodiment of the present disclosure;

FIG. 16B is a side cross-sectional view of the disinfecting cap shown in FIG. 16A with a fluid path element connected to the disinfecting cap;

FIG. 17A is a perspective cross-sectional view of a disinfecting cap for use with a fluid path element of a fluid path set in accordance with another embodiment of the present disclosure;

FIG. 17B is a side cross-sectional view of the disinfecting cap shown in FIG. 17A with a fluid path element connected to the disinfecting cap;

FIG. 17C is a perspective cross-sectional view of a cylindrical fluid volume feature of the disinfecting cap shown in FIG. 17A;

FIG. 18A is a perspective cross-sectional view of a disinfecting cap engaged with a fluid path element of a fluid path set in accordance with another embodiment of the present disclosure;

FIG. 18B is a side cross-sectional view of the disinfecting cap shown in FIG. 18A without the fluid path element connected to the disinfecting cap;

FIG. 19A is a perspective cross-sectional view of a disinfecting cap for use with a fluid path element of a fluid path set in accordance with another embodiment of the present disclosure;

FIG. 19B is a perspective cross-sectional view of the disinfecting cap shown in FIG. 19A without a compressible absorbent material;

FIG. 20A is a perspective cross-sectional view of a disinfecting cap for use with a fluid path element of a fluid path set in accordance with another embodiment of the present disclosure;

FIG. 20B is a perspective cross-sectional view of the disinfecting cap shown in FIG. 20A without a compressible absorbent material;

FIG. 20C is a perspective cross-sectional view of the disinfecting cap shown in FIG. 20A with a fluid path element shown connected to the disinfecting cap in a first position;

FIG. 20D is a perspective cross-sectional view of the disinfecting cap shown in FIG. 20A with a fluid path element shown connected to the disinfecting cap in a second position;

FIG. 21 is a perspective cross-sectional view of a disinfecting cap for use with a fluid path element of a fluid path set in accordance with another embodiment of the present disclosure;

FIG. 22A is a perspective cross-sectional view of a disinfecting cap for use with a fluid path element of a fluid path set in accordance with another embodiment of the present disclosure;

FIG. 22B is a perspective cross-sectional view of the disinfecting cap shown in FIG. 22A without a compressible absorbent material;

FIG. 23A is a side cross-sectional view of a disinfecting cap for use with a fluid path element of a fluid path set in accordance with another embodiment of the present disclosure;

FIG. 23B is a perspective view of a compressible absorbent material for use with the disinfecting cap shown in FIG. 23A;

FIG. 23C is a side cross-sectional view of a disinfecting cap for use with a fluid path element of a fluid path set in accordance with another embodiment of the present disclosure;

FIG. 23D is a perspective view of a compressible absorbent material and sealing insert for use with the disinfecting cap shown in FIG. 23C;

FIG. 24A is a side cross-sectional view of a disinfecting cap for use with a fluid path element of a fluid path set in accordance with another embodiment of the present disclosure;

FIG. 24B is a perspective view of a compressible absorbent material of the disinfecting cap shown in FIG. 24A;

FIG. 24C is a side cross-sectional view of the disinfecting cap shown in FIG. 24A with a fluid path element connected to the disinfecting cap;

FIGS. 24D-24E are perspective cross-sectional views of alternative configurations of the disinfecting cap shown in FIG. 24A;

FIG. 24F is a front view of the disinfecting cap shown in FIG. 24D;

FIG. 25 is a side cross-sectional view of a disinfecting cap for use with a fluid path element of a fluid path set in accordance with another embodiment of the present disclosure;

FIG. 26A is a side cross-sectional view of a disinfecting cap for use with a fluid path element of a fluid path set in accordance with another embodiment of the present disclosure;

FIG. 26B is a perspective cross-sectional view of the disinfecting cap shown in FIG. 26A without a compressible absorbent material;

FIG. 27A is a perspective cross-sectional view of a disinfecting cap for use with a fluid path element of a fluid path set in accordance with another embodiment of the present disclosure;

FIG. 27B is a perspective cross-sectional view of an alternative configuration of the disinfecting cap shown in FIG. 27A, with a fluid path element shown connected to the disinfecting cap in a first position;

FIG. 27C is a perspective cross-sectional view of the disinfecting cap shown in FIG. 27B with a fluid path element shown connected to the disinfecting cap in a second position;

FIG. 28A is a perspective view of a disinfecting cap for use with a fluid path element of a fluid path set in accordance with another embodiment of the present disclosure;

FIG. 28B is a side perspective view of the disinfecting cap shown in FIG. 28A;

FIG. 28C is a side cross-sectional view of the disinfecting cap shown in FIG. 28A;

FIG. 29 is a side cross-sectional view of a disinfecting cap for use with a fluid path element of a fluid path set in accordance with another embodiment of the present disclosure;

FIG. 30A is a side cross-sectional view of a disinfecting cap for use with a fluid path element of a fluid path set in accordance with another embodiment of the present disclosure;

FIG. 30B is a perspective view of an elastic reservoir for use with the disinfecting cap shown in FIG. 30A;

FIG. 31A is a side cross-sectional view of a disinfecting cap for use with a fluid path element of a fluid path set in accordance with another embodiment of the present disclosure;

FIG. 31B is a perspective cross-sectional view of the disinfecting cap shown in FIG. A without the compressible absorbent material;

FIG. 32A is a perspective cross-sectional view of a disinfecting cap for use with a fluid path element of a fluid path set in accordance with another embodiment of the present disclosure;

FIG. 32B is an exploded side cross-sectional view of a disinfecting cap of FIG. 32A.

FIG. 33A is a side cross-sectional view of a disinfecting cap for use with a fluid path element of a fluid path set in accordance with another embodiment of the present disclosure;

FIG. 33B is a perspective cross-sectional view of an alternative configuration of the disinfecting cap shown in FIG. 33A;

FIG. 34A is a perspective view of a strip having a plurality of disinfecting caps in accordance with another embodiment of the present disclosure; and

FIG. 34B is a perspective view of packaging for receiving a plurality of strips of disinfecting caps shown in FIG. 34A.

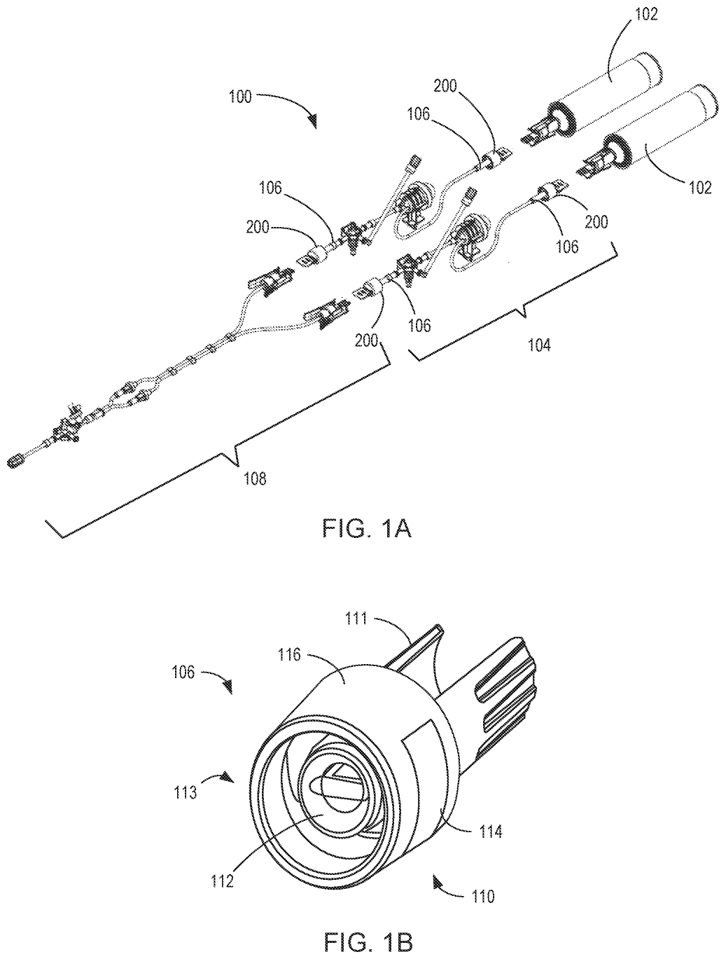

In FIGS. 1-34B, like characters refer to the same components and elements, as the case may be, unless otherwise stated.

### DETAILED DESCRIPTION

The following description is provided to enable those skilled in the art to make and use the described aspects contemplated for carrying out the disclosure. Various modifications, equivalents, variations, and alternatives, however, will remain readily apparent to those skilled in the art. Any and all such modifications, variations, equivalents, and alternatives are intended to fall within the spirit and scope of the present disclosure.

As used herein, the singular form of “a”, “an”, and “the” includes plural referents unless noted otherwise. With respect to the use of any plural and/or singular terms herein, those having skill in the art can translate from the plural to the singular and/or from the singular to the plural as is appropriate to the context and/or application. The various singular/plural permutations are not expressly set forth herein for sake of clarity.

Spatial or directional terms, such as “left”, “right”, “inner”, “outer”, “above”, “below”, and the like, relate to the embodiments or aspects as shown in the drawing figures and are not to be considered as limiting as the embodiments or aspects can assume various alternative orientations.

All numbers used in the specification and claims are to be understood as being modified in all instances by the term “about”. By “about” is meant plus or minus twenty-five percent of the stated value, such as plus or minus ten percent of the stated value. However, this should not be considered as limiting to any analysis of the values under the doctrine of equivalents.

Unless otherwise indicated, all ranges or ratios disclosed herein are to be understood to encompass the beginning and ending values and any and all subranges or sub ratios subsumed therein. For example, a stated range or ratio of “1 to 10” should be considered to include any and all subranges or sub ratios between (and inclusive of) the minimum value of 1 and the maximum value of 10; that is, all subranges or sub ratios beginning with a minimum value of 1 or more and ending with a maximum value of 10 or less. The ranges and/or ratios disclosed herein represent the average values over the specified range and/or ratio.

The term “includes” is synonymous with “comprises”. The terms “first”, “second”, and the like are not intended to refer to any particular order or chronology, but refer to different conditions, properties, or elements.

All documents referred to herein are “incorporated by reference” in their entirety.

The term “at least” is synonymous with “greater than or equal to.” The term “not greater than” is synonymous with “less than or equal to.” Some non-limiting embodiments or aspects may be described herein in connection with thresholds. As used herein, satisfying a threshold may refer to a value being greater than the threshold, higher than the threshold, greater than or equal to the threshold, less than the threshold, lower than the threshold, less than or equal to the threshold, equal to the threshold, etc.

In some instances, one or more components may be referred to herein as “configured to,” “operative,” “adapted,” etc. Those skilled in the art will recognize that “configured to” can generally encompass active-state components and/or inactive-state components and/or standby-state components, unless context requires otherwise.

Some aspects may be described using the expression “coupled” and “connected” along with their derivatives. It should be understood that these terms are not intended as synonyms for each other. For example, some aspects may be described using the term “connected” to indicate that two or more elements are in direct physical or electrical contact with each other. In another example, some aspects may be described using the term “coupled” to indicate that two or more elements are in direct physical or electrical contact. The term “coupled,” however, also may mean that two or more elements are not in direct contact with each other, but still co-operate or interact with each other.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be understood to include the possibilities of “A” or “B” or “A and B.”

When used in relation to a component of a fluid delivery system, such as a fluid reservoir, a syringe, a connector, a dust cap, or a fluid line, the term “distal” refers to a portion of said component nearest to a patient. When used in relation to a component of an injector system, such as a fluid reservoir, a syringe, a connector, a disinfecting cap, or a fluid line, the term “proximal” refers to a portion of said component nearest to the injector of the injector system (i.e. the portion of said component farthest from the patient). When used in relation to a component of a fluid delivery system such as a fluid reservoir, a syringe, a connector, a disinfecting cap, or a fluid line, the term “upstream” refers to a direction away from the patient and towards the injector in relation to the normal flow of fluid of the injector system. When used in relation to a component of a fluid delivery system such as a fluid reservoir, a syringe, a connector, a disinfecting cap, or a fluid line, the term “downstream” refers to a direction towards the patient and away from the injector in relation to the normal flow of fluid of the fluid delivery system.

The term “radial” and related terms refer generally to a direction normal to a longitudinal axis of a syringe, a connector, a dust cap, or other component of an injector system However, it is to be understood that the disclosure may assume various alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects disclosed herein are not to be considered as limiting.

It is noted that any reference to “an embodiment”, “one aspect”, or “an aspect” means that a particular feature, structure, or characteristic described in connection with the embodiment or aspect is included in at least one embodiment or aspect. Thus, appearances of the phrases “in one embodiment”, “in one aspect”, or “in an aspect” in various places throughout the specification are not necessarily all referring to the same aspect or embodiment. Furthermore, the particular features, structures or characteristics may be combined in any suitable manner in one or more embodiments or aspects.

One skilled in the art will recognize that the herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

Before explaining the various aspects of the disinfecting cap and various features thereof in detail, it should be noted that the various aspects disclosed herein are not limited in their application or use to the details of construction and arrangement of parts illustrated in the accompanying drawings and description. Rather, the disclosed devices may be positioned or incorporated in other devices, variations, and modifications thereof, and may be practiced or carried out in various ways. Accordingly, aspects of the disinfecting cap and features disclosed herein are illustrative in nature and are not meant to limit the scope or application thereof. Furthermore, unless otherwise indicated, the terms and expressions employed herein have been chosen for the purpose of describing the various aspects of the syringe and syringe features for the convenience of the reader and are not to limit the scope thereof. In addition, it should be understood that any one or more of the components of the disinfecting cap and features, expressions thereof, and/or examples thereof, can be combined with any one or more of the other components, expressions thereof, and/or examples thereof, without limitation.

The present disclosure is drawn to a design of a disinfecting cap for use with a fluid path element associated with a powered fluid injector used in medical imaging procedures. According to various embodiments, certain medical imaging procedures may include injection of a contrast media or agent that highlights certain features in the medical image. Known as contrast enhanced medical imaging, the process generally involves injection of a contrast media with a suitable flushing agent, such as saline, prior to the imaging process. Powered fluid injectors have been used to control injection of the fluids and are typically designed with one or more, commonly two, syringes for holding and dispensing the contrast media, the flushing fluid, and other medical fluids administered prior to or during the imaging procedure. For example, U.S. Pat. No. 5,383,858 discloses a front-loading syringe and powered injector in pressure jacket and jacketless examples, which disclosure is incorporated by this reference. Common contrast enhanced medical imaging procedures include computed tomography (CT), magnetic resonance imaging (MR), positron emission tomography (PET, SPECT), and angiography (CV). Due to the viscosity and need to deliver volumes of contrast during a short period of time, to provide a “tight bolus,” certain injection procedures may be performed at high injection pressures, such as pressures up to 300 psi for CT and MR, and pressures up to 1200 psi for CV procedures. The injector may be configured to inject or dispense the fluid medium contained in the first, second, and/or further syringes in a controlled manner, such as may be employed in medical procedures such as angiography, CT, PET, and NMR/MRI. For example, U.S. Pat. No. 5,383,858 and PCT International Publication No. WO 2022/035791 disclose a front-loading syringes and powered injector in pressure jacket and jacketless examples, which disclosures are incorporated by this reference.

Fluid injection systems may include configurations for multi-patient injections where a portion of the disposable elements of the fluid path set, such as the pumping mechanism (syringe, components of a peristaltic pump system, etc.) and upstream components of the fluid path set are used over a series of injection procedures before disposal (multi-patient elements); and another portion of disposable elements of the fluid path set (single patient elements) which are used once with a single patient and then disposed. Suitable examples of multi-patient elements and single patient elements are described, for example, in U.S. Pat. Nos. 10,507,319 and 10,549,084 and International PCT Publication Nos. WO 2021/173743; WO 2022/119837; and WO 2022/182935, which disclosure is incorporated by this reference. Upon completion of a fluid injection procedure, the single patient elements are disconnected from the multi-patient elements of the fluid path and disposed; and a new, sterile single patient element is attached for the next injection procedure. Such an arrangement may reduce expenditures per fluid injection procedure and reduce the amount of medical waste produced by an injection suite. In between the injection procedures, care must be taken to ensure that the distal connection(s) of the multi-patient elements are not contaminated with microbial contaminants and are sterile when attaching the new single-patient element. Sterility of the single patient element is ensured by storage and removal from the associated sterile packaging immediately prior to connection with the multi-patient element. The present disclosure provides components that can be utilized with the multi-patient element to ensure that the connection features of the multi-patient elements are sterile when connected with the single patient element in preparation for a subsequent injection procedure.

With reference to FIG. 1A, an exemplary fluid path set 100 is shown in accordance with some embodiments. The fluid path set 100 may include at least one fluid reservoir 102, such as a syringe or peristaltic pump mechanism (not shown), that is connectable to a fluid injector for delivering fluid from the fluid reservoir 102 to a multi-patient fluid path set 104, and/or for filling the fluid reservoir 102 with fluid from a bulk fluid source. The multi-patient fluid path set 104 includes fluid path elements 106 for connecting to the fluid reservoir 102 at one end and a single-patient fluid path set 108 at an opposing end. The single-patient fluid path set 108 is configured to be disconnected from the multi-patient fluid path set 104 after each use and disposed; and a new, sterile single-patient fluid path set 108 is configured to be attached to the fluid path elements of the multi-patient fluid path set 104 for the next injection procedure. After a predetermined amount of time and/or a predetermined number of fluid delivery procedures, the multi-patient fluid path set 104 is configured to be disposed.

With reference to FIG. 1B, the fluid path element 106 of the multi-patient fluid path set 104 (shown in FIG. 1B) may include a body 110 having a lumen 112 extending therethrough configured for connection to a corresponding connector on the fluid reservoir 102 and/or the single-patient fluid path set 108. The body 110 has a proximal end 111 configured for connecting to tubing and a distal end 113 configured for connecting to the corresponding connector on the fluid reservoir 102 and/or the single-patient fluid path set 108, and which may be protected and disinfected by disinfecting cap 200, as described herein. In one example of the present disclosure, the body 110 is configured to be substantially cylindrical in shape. The body 110 may define at least one aperture 114 that extends through an outer skirt 116 surrounding an outer surface of the lumen 112. The outer skirt 116 may assist in maintaining sterility of the fluid path, for example by preventing inadvertent touching and contamination of the inner lumen 112 by a technician during manipulation of the fluid path element 106. In certain embodiments of the present disclosure, the body 110 may have no apertures defined on the outer skirt 116. Additional details of the construction of the fluid path element 106 are described in described in International PCT Application Publication No. WO 2021/168076, incorporated herein in its entirety by this reference.

Without intending to be limited by any particular theory, if is believed that microbial contamination, if it occurs, will be primarily on the outer “touchable” surfaces of the fluid path element 106 assembly and outer surfaces of the lumen 112 and not on the inner surfaces of the lumen 112. Touch contamination on the surfaces of the inner surfaces of the lumen 112 is unlikely due to the shrouding effects of the outer shroud 116 surrounding the lumen 112 and extending past the end surface of the lumen 112. However, environmental contamination may be possible with extended exposure time, for example by interaction with airborne contaminants or microbial migration. Surfaces outside the fluid pathway defined by the lumen 112 are unlikely to promote transfer of microbial contaminants to the lumen 112 during connection or disconnection. Disinfection of these surfaces, for example the outer surfaces of the lumen 112 and the inner surfaces of the fluid path element 106 wall by use of the disinfecting caps 200 described herein may prevent transfer of microbial contamination to the inner lumen 112 and the fluid contacting surfaces thereof.

The present disclosure provides embodiments of disinfecting caps 200 which are configured to be removably attached to the fluid path elements 106 of the multi-patient fluid path set 104 during shipping and/or after removal of the single patient fluid path set 108. The disinfecting caps 200 may perform several functions including, but not limited to: i) covering the fluid path elements 106 of the multi-patient fluid path set 104 to prevent inadvertent contamination, for example by contact of the fluid path elements 106 of the multi-patient fluid path set 104 with a contaminated surface (e.g., a surface in the injection suite or accidental contact with the hand of the technician); and disinfecting portions of the fluid path elements 106 of the multi-patient fluid path set 104 by contact with a disinfecting, antiseptic, and/or sterilizing fluid (e.g., isopropyl alcohol solutions, ethanol solutions, mixtures and aqueous solutions thereof, etc.) that is stored within or added to the disinfecting cap 200 prior to removal of the disinfecting cap 200 and connection with the single patient fluid path set 108. Ethanol solutions may be used as the have been shown to be effective antimicrobial agents and are compatible within the human blood stream, reducing the need to seal the inner lumen of the fluid path from contact with the disinfecting fluid. Isopropyl alcohol is known to be bactericidal, tuberculogical, fungicidal and virucidal. In certain embodiments, one or more sensors may be incorporated into the fluid injector to monitor the time of contact of the fluid path element with the disinfecting cap 200 to ensure that complete disinfection is achieved.

The disinfecting caps 200 may be configured for either standard connection features, such as a Luer-type connector, or may be engineered to interact with non-standard connection features, such as but not limited to connectors described in International PCT Application Publication Nos. WO 2021/168076; WO 2016/112163; WO 2015/106107; and WO 2006/060688, or non-standard syringe nozzles, such as described in WO 2019/055497, the disclosures of which are incorporated by this reference.

According to various embodiments, the disinfecting caps 200 of the present disclosure may include an absorbent material, for example a polymeric sponge, a cotton material, and the like, within an interior portion of the disinfecting cap 200, wherein the absorbent material is at least partially saturated with the disinfecting fluid. As the disinfecting cap 200 is engaged to the fluid path element 106 of the multi-patient fluid path set 104 and an activating surface is pressed by a user, the disinfecting cap and absorbent material is pressed against the multi-patient fluid path element 106, compressing the absorbent material, and causing the disinfecting fluid to contact at least a portion of one or more surfaces of the multi-patient fluid path element 106, thereby disinfecting the one or more surfaces. In certain embodiments, at least a portion of the sponge may comprise an open cell matrix to releasably trap the disinfecting fluid. In various embodiments, at least a portion of the sponge may comprise a closed cell matrix which is non-absorbent to the disinfecting fluid. For example, closed cell portions may be incorporated into parts of the sponge which contact the rim of lumen 112 to prevent release of disinfecting fluid into the interior of the lumen 112, whereas portions of the sponge that do not contact the areas abutting the lumen may include an open cell matrix to release disinfecting fluid to surfaces outside of the lumen upon compression.

In certain embodiments, the disinfecting cap 200 may comprise a fluid path sealing portion or protrusion that sealably engages with the lumen 112 of the multi-patient fluid path set 106 to prevent the disinfecting fluid from entering the lumen 112 and/or to prevent dripping of any fluid from the interior of the lumen 112. By preventing the disinfecting fluid from entering the lumen 112, contamination of the lumen and any medical fluid therein is avoided, for example, by preventing flushing of any microbial contaminants into the lumen 112 when the disinfecting fluid flows through the passageways and contacts the at least a portion of one or more surfaces of the multi-patient fluid path set 104. For example, the sealing portion or protrusion may include a sealable material (e.g., silicone, polymeric material, a closed cell foam, etc.) that engages the rim of the lumen and seals and prevents ingress of disinfecting fluid into the lumen 112.

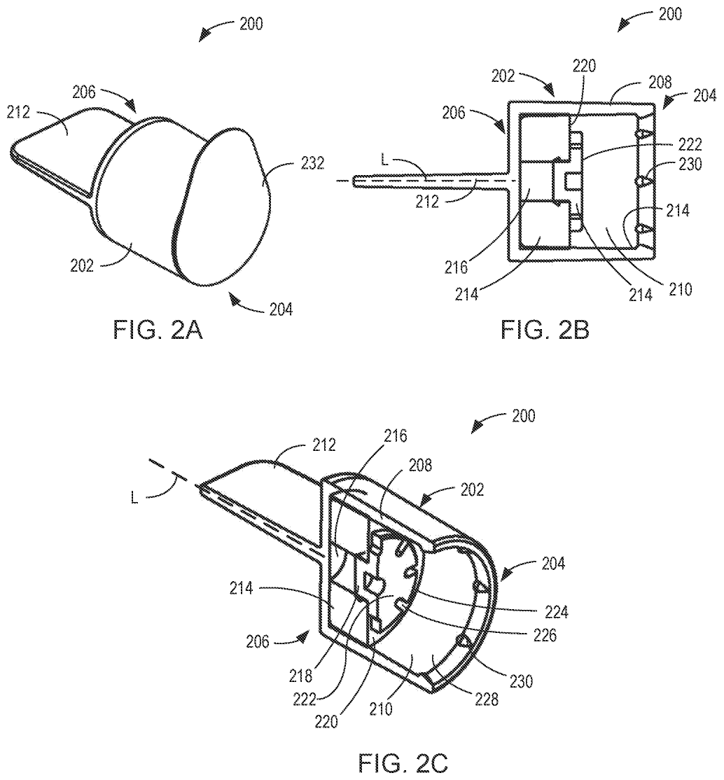

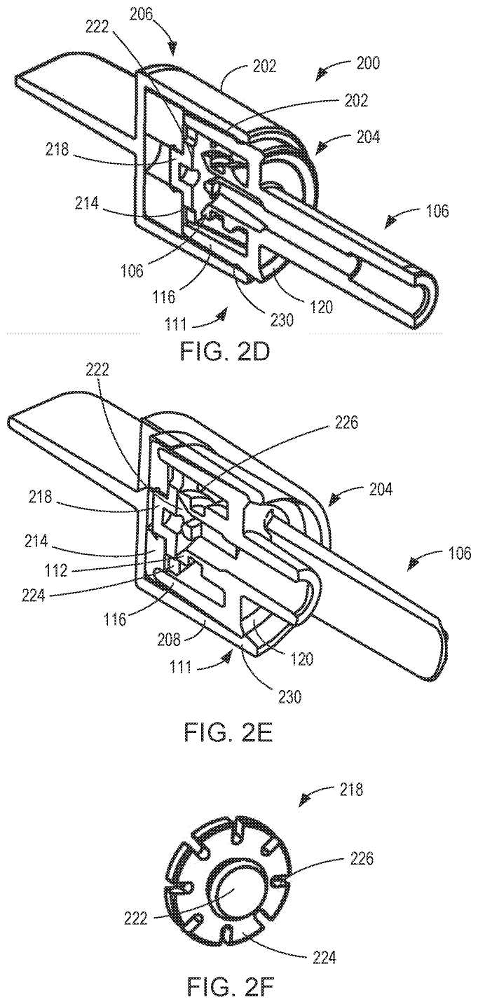

With reference to FIGS. 2A-2C, the disinfecting cap 200 is shown in accordance with one embodiment of the present disclosure. The disinfecting cap 200 may include a housing 202 configured to fit over at least a portion of the fluid path element 106 (shown in FIG. 1B). In some embodiments, the housing 202 may be made from a medical-grade plastic material. The housing 202 may include an open proximal end 204, a closed distal end 206, and a sidewall 208 extending between the open proximal end 204 and the closed distal end 206, forming an interior volume 210 (shown in FIGS. 2B-2C). In some embodiments, the housing 202 may have a shape that corresponds to a shape of at least a portion of the fluid path element 106. For example, the housing 202 may be configured and engineered to fit over at least a portion of the fluid path element 106, such as the lumen 112 and the outer skirt 116. The exterior of the closed distal end 206 may include a gripping element 212, such as a tab, for handling by a user to allow ready attachment and removal of the disinfecting cap 200 to the fluid path element 106. The gripping element 212 may protrude distally from the closed distal end 206. In some embodiments, the gripping element 212 may be formed on an outer surface of the housing 202, such as sidewall 208. For example, the gripping element 212 may be one or more protrusions that protrude radially outward from an outer surface of the housing 202.

With reference to FIGS. 2B-2C, a compressible absorbent material 214 may be disposed within the interior volume 210 of the housing 202. In some embodiments, the compressible absorbent material 214 may abut or be connected to the closed distal end 206. The compressible absorbent material 214 may be at least partially saturated with a disinfecting fluid, as described herein. In some embodiments, the compressible absorbent material 214 may have an annular shape with a central opening 216. In other embodiments, the compressible absorbent material 214 is made in a cylindrical shape to fill at least a portion of the interior volume 210. Compressible absorbent material 214 may be made from a sponge material (open and/or closed cell), a cotton material, other medical grade absorbent material, and the like.

Other embodiments of the disinfecting cap 200 may be provided in a dry, sterilized form and the disinfecting fluid may be added to the disinfecting cap 200 just prior to or once the disinfecting cap 200 is attached to the fluid path element 106, for example from a bulk disinfecting fluid source. In this manner, issues associated with sealing the disinfecting cap 200 with the fluid and the potential of the fluid drying out may be avoided.

With reference to FIGS. 2D-2E, as the disinfecting cap 200 is engaged to the fluid path element 106 of the multi-patient fluid path set 104 in a first position (FIG. 2D), and the fluid path element 106 is urged toward the closed distal end 206, the disinfecting cap 200 and compressible absorbent material 214 is pressed against the multi-patient fluid path element 106 in a second position (FIG. 2E), thereby compressing the compressible absorbent material 214, and causing the disinfecting fluid to contact at least a portion of one or more surfaces of the multi-patient fluid path element 106, thereby disinfecting the one or more surfaces. While FIGS. 2D and 2E illustrate different disinfecting caps 200, the engagement mechanism is the same in both disinfecting caps 200.

With reference to FIGS. 2B-2C, the disinfecting cap 200 may include a slidable insert 218 proximally adjacent to the compressible absorbent material 214 within the interior volume 210. For example, the slidable insert 218 may abut a proximal surface 220 of the compressible absorbent material 214. The slidable insert 218 may include a fluid path sealing surface 222 configured to engage with a distal surface 118 (shown in FIG. 1B) of the lumen 112 of the fluid path element 106. As shown in FIGS. 2D-2E, the fluid path sealing surface 222 is configured to seal the lumen 112 of the fluid path element 106, thereby preventing fluid communication and ingress of the disinfecting fluid into the lumen 112 and preventing flow of a medical fluid within the lumen 112 into the interior volume 210 of the disinfecting cap 200. In certain embodiments, the fluid path sealing surface 222 may comprise a rubber, pliable plastic, closed cell sponge, or silicone material configured to create a fluid tight seal with the lumen 112 of the fluid path element 106.

With reference to FIG. 2C, the slidable insert 218 may further include a circumferential flange 224 extending radially around the fluid path sealing surface 222. The circumferential flange 224 may include one or more passageways 226, such as slots or holes, extending through the slidable insert 218 providing fluid communication therethrough. In some embodiments, the one or more passageways 224 may be made of or coated with a flexible material that retains the passageways in the normally closed position (no fluid communication therethrough) and then are configured to deform and open under fluid pressure, such as when the fluid path element 106 is urged toward the closed distal end 206, to allow fluid communication between the disinfecting fluid in the compressible absorbent material 214 and the fluid path element 106. Alternatively, the passageways 226 may include a cover material, for example by flashing over the hole, or by adhesively attaching a membrane, that will at least partially release or break when sufficient pressure is applied by the disinfecting fluid.

When the slidable insert 218 is engaged with the distal end of the fluid path element 106, for example by pressing the disinfecting cap 200 onto the fluid path element 106 or moving the disinfecting cap 200 from a non-activated, first position, where the disinfecting cap 200 200 simply covers the distal end of the fluid path element 106 to an activated, second position, where the disinfecting cap 200 is further pressed against the distal end of the fluid path element 106, the circumferential flange 224 is pressed against the compressible absorbent material 214, thereby compressing the compressible absorbent material 214 (see FIG. 2E), which releases the disinfecting fluid from the compressible absorbent material 214 through the one or more passageways 226 and into contact with surfaces of the fluid path element 106, allowing disinfection of the contacted surfaces.

At least partial compression of the compressible absorbent material 214 is configured to release the disinfecting fluid from the compressible absorbent material 214 and the disinfecting fluid flows through the one or more passageways 226 of the circumferential flange 224 and contacts one or more surfaces of the fluid path element 106, such as an exterior surface of the lumen 112 and the inner and outer surfaces of the outer skirt 116. Contact of the disinfecting fluid with the various surfaces and sealing elements (e.g., O-rings, etc.) of the fluid path element 106 for an appropriate time, such as from several seconds up to 7 days, effectively disinfects the contacted surfaces of any microbial contaminant, such as germs, bacteria, viral particles, bodily fluids of previous patients, and other disease-causing pathogens. In certain embodiments, the disinfecting cap 200 and corresponding compressible absorbent material 214 may be rotated around the longitudinal axis of the fluid path element 106, resulting in a rubbing action to further assist with disinfection of the contacted surfaces. After the disinfecting cap 200 has been in disinfecting engagement with the fluid path element 106 for the predetermined of time and when the technician is prepared to begin the next injection procedure, the disinfecting cap 200 may be removed, for example by gripping the gripping element 212 and pulling the disinfecting cap 200 from the fluid path element 106, and a new single patient fluid path set 108 (shown in FIG. 1A) may be connected to the disinfected distal end of the multi-patient fluid path element 106.

According to an alternative embodiment, the disinfecting fluid may be housed within the disinfecting cap 200 without the compressible absorbent material 214, such that the disinfecting fluid is sealably retained by the circumferential flange of the sliding insert 224. In such embodiments, the circumferential flange 224 may be sealably connected to an inner surface 228 of the sidewall 208. Pressing the disinfecting cap 200 against the distal end of the fluid path element 106 creates pressure on the disinfecting fluid to a point where the pressure overcomes the seal between the circumferential flange 224 and the inner surface 228 of the sidewall 208 of the disinfecting cap 200. Disinfecting fluid may then flow past the sliding insert 218 and contact the fluid path element 106 as described herein. Further, as the insert 218 is pressed against the distal surface of the fluid path element 106, the fluid path sealing surface 222 creates a fluid-tight seal with the lumen 112, preventing fluid communication between the interior volume 210 of the disinfecting cap 200 and the lumen 112.

With reference to FIGS. 2B-2C, the inner surface 228 of the disinfecting cap 200 may include one or more radially inwardly protruding projections 230 or retaining bumps at the proximal end 204. The one or more radially inwardly protruding projections 230 may be configured for removably engaging with a corresponding engagement feature 120, such as a flange, a top surface, an at least partial lip, and/or at least partial circumferential groove around the proximal end 111 of fluid path element 106, or one or more indentations on a surface of the outer skirt 116 of the fluid path element 106 shown in FIGS. 2D-2E. Engagement of the one or more projections 230 with the corresponding engagement feature 120 on the fluid path element 106 may releasably retain the disinfecting cap 200 on the fluid path element 106 and prevent inadvertent removal of the disinfecting cap 200, for example by bumping or the effects of gravity during storage or shipping.

The interaction between the one or more radially inwardly protruding projections 230 on the disinfecting cap 200 and the corresponding engagement feature 230 on the fluid path element 106 may be configured to provide an auditory and/or visual indicator to indicate to the technician that the disinfecting cap 200 is secured to the fluid path element 106 and/or that the disinfecting cap 200 has been moved to the second, activated state and the fluid path element 106 has been contacted with the disinfecting fluid. The features for producing the auditory and/or visual indicator may be located on the disinfecting cap 200, the fluid path element 106, or may be located on both where they are configured to interact as the disinfecting cap 200 is attached and/or moved to the second, activated state. The auditory and/or visual indicators may further include one or more colors, lines, permanent deformations, audible clicks, or snaps, etc. to indicate to the technician that the disinfecting fluid has been dispensed and that the fluid path element 106 has been disinfected.

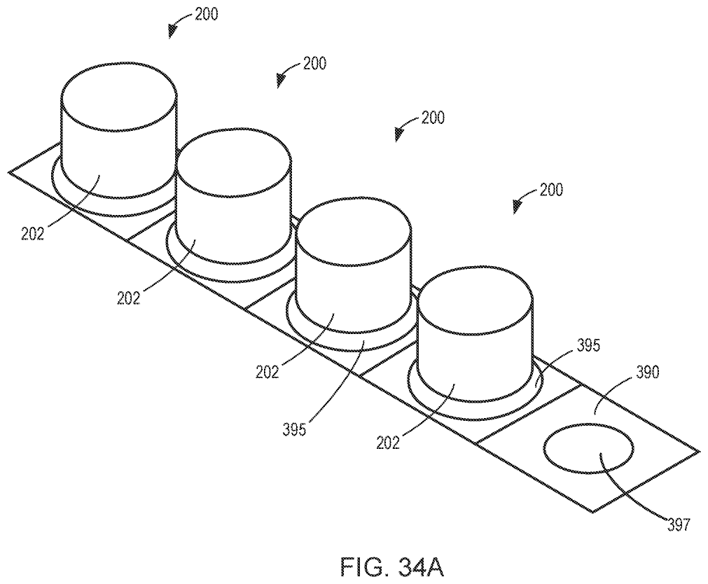

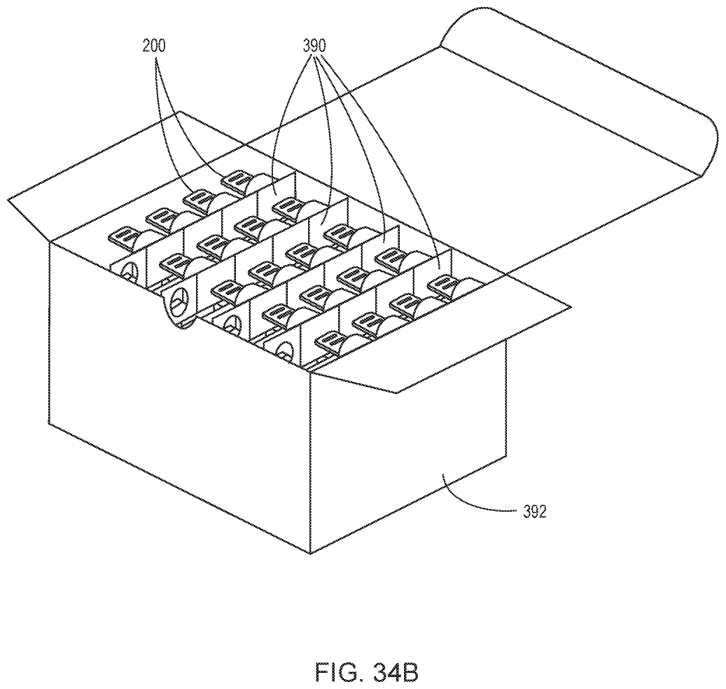

With reference to FIG. 2A, the disinfecting cap 200 may further include a seal 232. In some embodiments, the seal 232 may extend across the open proximal end 204 to enclose the interior volume 210 (shown in FIG. 2B). The seal 232 may be an adhesive and/or frangible seal, removably attached to and covering the open proximal end 204 of the disinfecting cap 200. The seal 232 allows for shipping and storage of the disinfecting cap 200 without loss of (e.g., evaporation) and or contamination of the disinfecting fluid in the interior volume 210 of the housing 202. The seal 232 may be a single seal, i.e., a seal that only covers a single disinfecting cap 200, or may be a multi-cap seal, for example, an extended rectangular or square surface having a plurality of sealing sections, for storing and sealing a plurality of disinfecting caps 200, as discussed herein with reference to FIG. 34A. The plurality of disinfecting caps 230 may be removed one-by-one, as needed, from the multi-cap seal 232 without affecting the sterility or condition of the remaining disinfecting caps 200.

The disinfecting cap 200 may be connectable to the fluid path set 106 in a first, inactive state, wherein the disinfecting fluid is not in contact with surfaces of the fluid path element 106, and a second active state, wherein the disinfecting fluid is in contact with the surfaces of the fluid path element 106. In some embodiments, the disinfecting cap 200 may transition from the inactive state to the active state by movement of the disinfecting cap 200 relative to the fluid path element 106, such as by an axial movement along the longitudinal axis L (shown in FIGS. 2B and 2C) and/or by a rotational movement about the longitudinal axis L.

In certain embodiments, the disinfecting cap 200 may be configured for twisting about the longitudinal axis L after pushing to engage the disinfecting cap 200 and the compressible absorbent material 214 with the fluid path element 106 to provide a scrubbing action between the compressible absorbent material 214 and various surfaces of the fluid path element 106 to further contact the disinfecting fluid with surfaces of the fluid path element 106 and provide increased disinfecting action. Twisting may be accomplished by rotating the gripping element 212 manually during or after engaging the disinfecting cap 200 with the fluid path element 106.

After activation, the disinfecting cap 200 may be left engaged with the fluid path element 106 in the activated state for a time sufficient to disinfect all contacted surfaces from any microbial or pathogenic materials. The disinfecting cap 200 may also act as a dust cap, preventing contamination of the distal end 113 of the fluid path element 106 with dust particles that may be floating in the air in the injection suite. The disinfecting cap 200 may be left in place until the next patient and the injector are prepared for the subsequent patient's injection procedure, at which time they can be removed and the disinfected/sterilized distal end of the multi-patient fluid path element 106 may be engaged with the proximal end of the single-patient fluid path set 108 (shown in FIG. 1A).

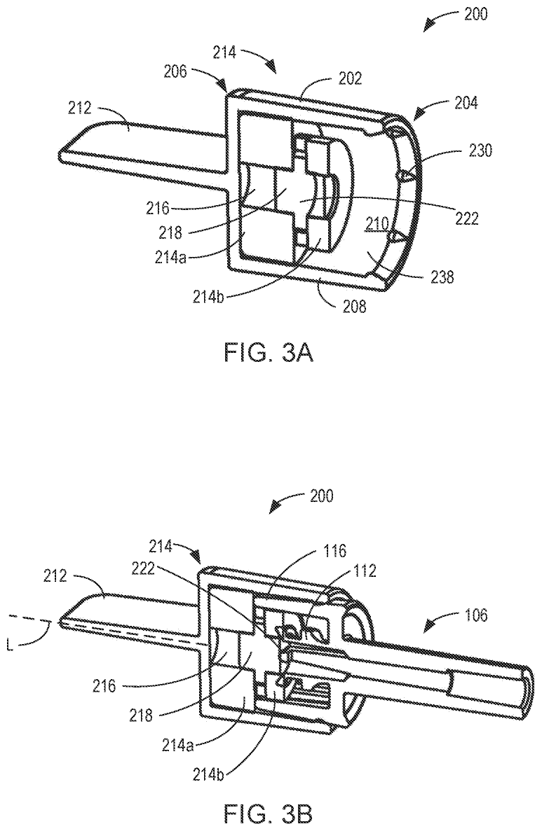

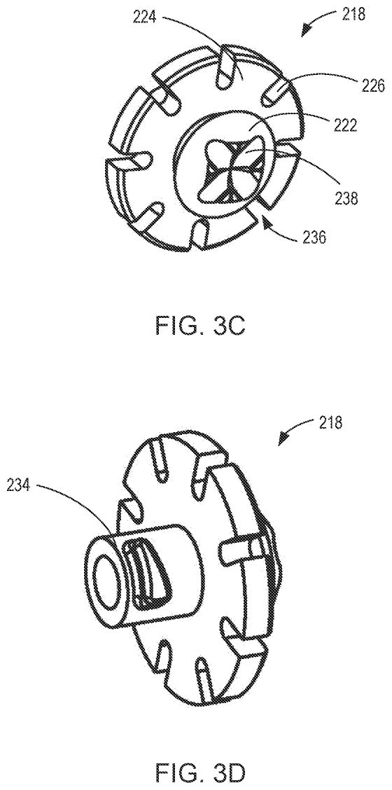

With reference to FIGS. 3A-34B, the disinfecting cap 200 is shown in accordance with additional embodiments of the present disclosure. The components of the disinfecting cap 200 shown in FIGS. 3A-34B are substantially similar or identical to the components of the disinfecting cap 200 described herein with reference to FIGS. 2A-2F. Accordingly, reference numerals in FIGS. 3A-34B are used to illustrate identical components of the corresponding reference numerals in FIGS. 2A-2F. As the previous discussion regarding the disinfecting cap 200 generally shown in FIGS. 2A-2F is applicable to the disinfecting cap 200 shown in FIGS. 3A-34B, only the relative differences between disinfecting caps 200 are discussed hereinafter.

With reference to FIGS. 3A-3B, the disinfecting cap 200 may include a dual compressible absorbent material arrangement having a compressible absorbent material 214 on both sides of the slidable insert 218. Specifically, a first compressible absorbent material 214a is provided on a distal side of the insert 218 and a second compressible absorbent material 214b is provided on a proximal side of the insert 218. The first compressible absorbent material 214a acts to release disinfecting fluid to contact various outer surfaces of the fluid path element 106, such as surfaces of the outer skirt 116 of fluid path element 106. The second compressible absorbent material 214b has a central opening 216 which is configured to receive the lumen 112 of the fluid path element 106 such that the lumen 112 can contact the fluid path sealing surface 222 of the slidable insert 218 to seal the lumen 112 from the interior volume 210 of the disinfecting cap 200. As shown in FIG. 3B, the second compressible absorbent material 214b is configured to be received inside the outer skirt 116 of the fluid path element 106. In this manner, rotation of the disinfecting cap 200 about the longitudinal axis L relative to the fluid path element 106 will scrub an inner surface of the outer skirt 116 and outer surface of lumen 112 via the second compressible absorbent material 214b.

With reference to FIGS. 3C-3D, the insert 218 may have a threaded distal end 234 that is configured to threadably engage with the housing 202 of the disinfecting cap 200. For example, the disinfecting cap 200 may include an inner threaded portion configured to engage threaded distal end 234 of the slidable insert 218 such that, as the disinfecting cap 200 is rotated about the longitudinal axis L, the slidable insert 218 moves distally toward the closed distal end 206 to compress the compressible absorbent material 214 and release the disinfecting fluid. A proximal end 236 of the insert 218 may have a key 238 that is configured to engage with the fluid path element 106, such as the lumen 112. In this manner, when the disinfecting cap 200 is connected to the fluid path element 106, corresponding features on the end of the lumen 112 engages the key 238 such that rotation of the disinfecting cap 200 about the longitudinal axis L moves the sliding insert 218 in the distal direction via the threaded engagement between the threaded distal end 234 and the housing 202. Such distal movement compresses the compressible absorbent material 214 to release the disinfecting fluid to disinfect fluid path element 106.

In alternative embodiments, the threaded portion may be on the slidable insert 218 and may be configured to interact with associated protrusions on an inner surface portion of the disinfecting cap 200 to screw the disinfecting cap 200 onto the fluid path element 106. A rear portion of the slidable insert 218 may include one or more rotation keys that are configured to engage with corresponding features on the end of the fluid path element 106, such as a fluid diverter element (see, e.g., International PCT Application No. PCT/US2021/018523), to hold the slidable insert 218 in a non-rotational position as the disinfecting cap 200 is rotated.

In another embodiment, the disinfecting cap 200 may include a slidable insert 218 with a push-to-screw configuration. According to this embodiment, the act of pushing the disinfecting cap 200 onto the fluid path element 106 in a direction of the longitudinal axis L causes the slidable insert 218 to engage with a threaded portion of the disinfecting cap 200 and self-screw/rotate the slidable insert 218 during the engagement process, thereby compressing the compressible absorbent material 214 and releasing the disinfecting fluid from the compressible absorbent material 214 to disinfect the surfaces of the fluid path element 106.

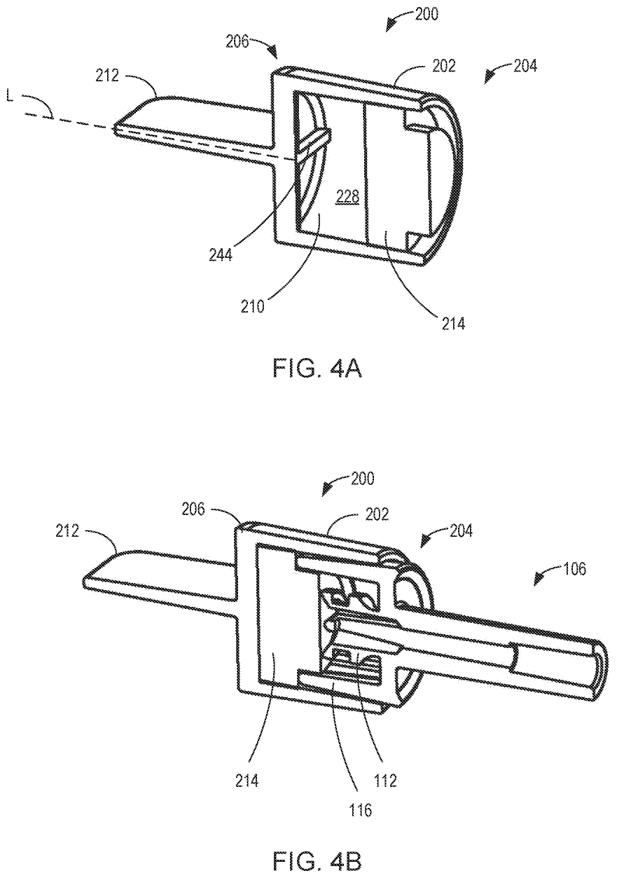

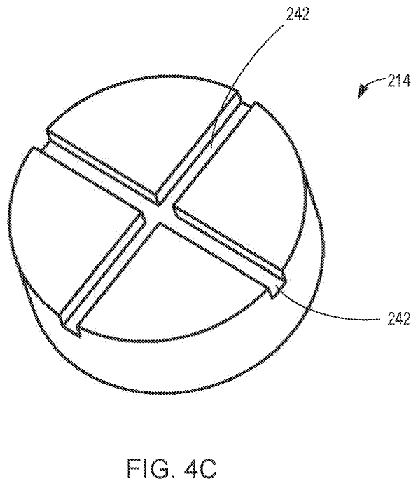

With reference to FIGS. 4A-4C, the disinfecting cap 200 may have a keyed anti-rotation compressible absorbent material 214. For example, the disinfectant-saturated compressible absorbent material 214 may have one or more anti-rotation slots 242 (shown in FIG. 4C) configured to engage corresponding one or more anti-rotation protrusions 244 on the inner surface 228 of the housing 202 (shown in FIG. 4A). The one or more anti-rotation slots 242 may be configured to receive the one or more anti-rotation protrusions 244 such that the compressible absorbent material 214 rotates with rotation of the disinfecting cap 200 about the longitudinal axis L (shown in FIG. 4A). In some embodiments, the location of the one or more anti-rotation slots 242 and the one or more anti-rotation protrusions 244 may be reversed such that the one or more anti-rotation slots 242 are provided on the inner surface 228 of the housing 202 and the one or more anti-rotation protrusions 244 are provided on the compressible absorbent material 218.

Because the compressible absorbent material 218 is keyed to and rotates with the disinfecting cap 200, as the disinfecting cap 200 is pushed toward the fluid path element 106, the keyed elements (i.e., the one or more anti-rotation slots 242 and the one or more anti-rotation protrusions 244) engage so that the absorbent material 218 rotates with the disinfecting cap 200. In this manner, as the disinfecting cap 200 is rotated about the longitudinal axis L, the compressible absorbent material 218 scrubs one or more surfaces of the fluid path element 106 while releasing disinfecting fluid, thereby improving the disinfecting action. While the keyed arrangement is described as including the one or more anti-rotation slots 242 and the one or more anti-rotation protrusions 244, other complementary keyed features may be incorporated into the disinfecting cap 200 and the compressible absorbent material 218 and still be within the scope of the present embodiment.

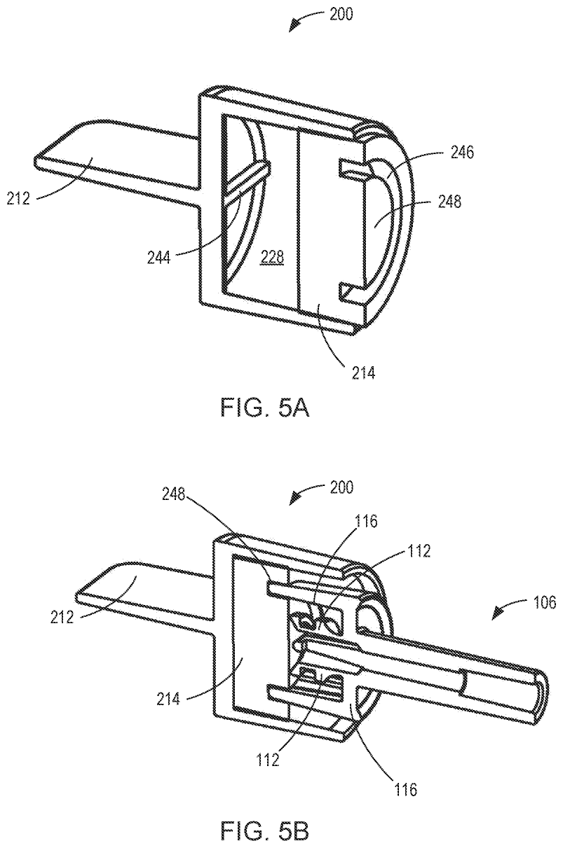

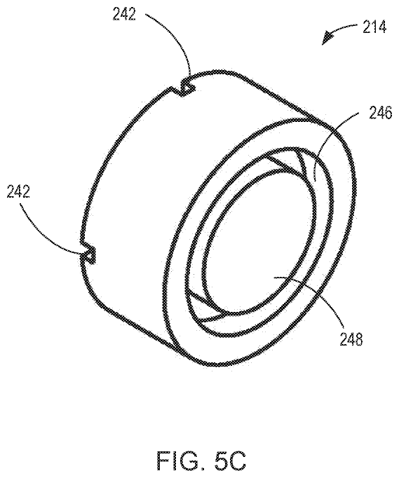

With reference to FIG. 5A, the various embodiments of the compressible absorbent material 218 may have a circumferential groove 246 recessed into a proximal surface 248 (illustrated with reference to the embodiment of FIGS. 4A-4C, but not limited thereto). The circumferential groove 246 may be configured to engage the distal rim of the outer skirt 116 of the fluid path element 106 (FIG. 5B) such that the compressible absorbent material 218 engages both the interior and exterior surfaces of the outer skirt 116. In certain embodiments, compressible absorbent material 218 may be keyed to engage with the inner surface 228 of the housing 202, as described herein, such that the compressible absorbent material 218 rotates with the disinfecting cap 200 during the disinfecting process so that the engaged surfaces of the fluid path element 106 are scrubbed and disinfected. In this embodiment, both the interior and exterior surfaces of the outer skirt 116 are contacted with the disinfectant fluid and are effectively sterilized during the scrubbing process due to contact with the compressible absorbent material 218. Alternatively, in another embodiment, the compressible absorbent material 218 having the circumferential groove 246 may be used as a stand-alone disinfecting element without the keyed connection with the housing 202. According to this alternative embodiment, the user would simply engage the compressible absorbent material 218 with the fluid path element 106 and rotate the absorbent element to scrub and disinfect the inner and outer surfaces of the outer skirt 116 of the fluid path element 106.

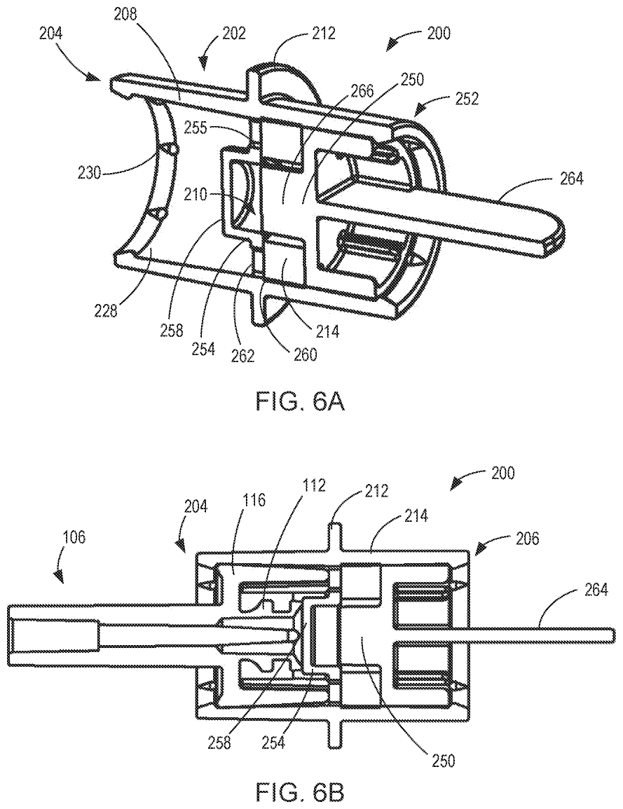

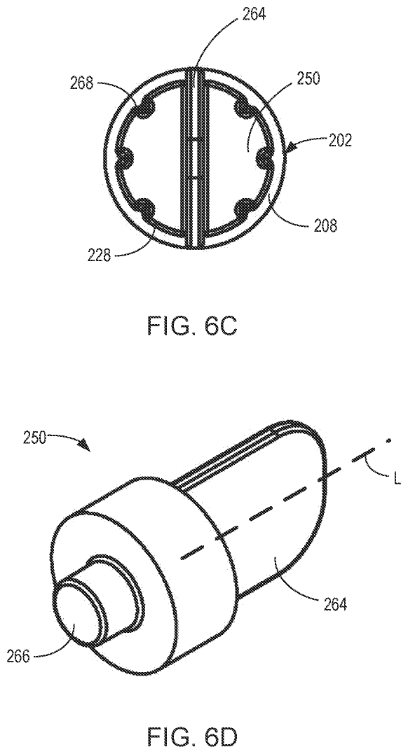

With reference to FIGS. 6A-6D, and according to another embodiment of the present disclosure, the disinfecting cap 200 may include a slidable plunger 250 that is movable relative to the housing 202 in a direction of the longitudinal axis L (shown in FIG. 6A) to compress the compressible absorbent material 218 and release the disinfecting fluid therefrom. The disinfecting cap 200 may include a housing 202 configured to fit over at least a portion of the fluid path element 106, wherein the housing 202 has the open proximal end 204, an open distal end 252, and the sidewall 208 extending between the open proximal end 204 and the open distal end 252. The gripping element 212 is illustrated as a flange that extends radially outward from at least a portion of an outer surface of the sidewall 208 of the housing 208 to allow a user to attach, activate, and remove the disinfecting cap 200 by gripping the gripping element 212. Other configurations of gripping element 212 are also considered

With continued reference to FIG. 6A, an inner flange 254 may extend radially inward from the inner surface 228 of the housing 202 into the interior volume 210. The inner flange 254 may include one or more fluid passageways 255 extending therethrough to permit a flow of the disinfecting fluid from a distal side 260 of the inner flange 254 to a proximal side 262 of the inner flange 254. In some embodiments, the inner flange 254 may include a sealing surface 258 on the proximal side 262. In certain embodiments, the sealing surface 258 may be on a proximally extending protrusion 256 located at a center portion of the inner flange 254 and surrounded by the one or more fluid passageways 255. The sealing surface 258 may be configured for sealing the lumen 112 of the fluid path element 106 to prevent ingress of any of the disinfecting fluid into an interior volume of the lumen 112 when the disinfecting cap 200 is connected to the fluid path element 106.

With continued reference to FIG. 6A, the disinfecting cap 200 may have the compressible absorbent material 218 between the proximal side 262 of the inner flange 254 and the slidable plunger 250. As the slidable plunger 250 is moved axially in a direction toward the proximal side 262 of the inner flange 254, the compressible absorbent material 218 is compressed to thereby release the disinfectant fluid therefrom. The disinfectant fluid can then flow through the one or more fluid passageways 255 to come into contact with the fluid path element 106. One or more radially inwardly protruding projections 230 or retaining bumps may be provided on the inner surface 228 of the housing 202 at the open proximal end 204 to interact with corresponding features or grooves on the fluid path element 106, as described herein with reference to FIGS. 2A-2F.

According to another embodiment, the compressible absorbent material 218 may be positioned on each side of the inner flange 254. For example, the compressible absorbent material 218 may have a first compressible absorbent material 218a on the distal side 260 of the inner flange 254 and a second compressible absorbent material 218b on the proximal side 262 of the inner flange 254. This arrangement of the first and second compressible absorbent materials 218a, 218b is similar to what is described herein with reference to FIGS. 3A-3B. The second compressible absorbent material 218b abutting the fluid path element 106 may have the circumferential groove 246 to engage both the interior features and exterior surface of the outer skirt 116, as described herein with reference to FIGS. 5A-5C.

With reference to FIGS. 6C-6D, the slidable plunger 250 may include a pressing surface 264 configured for allowing a user to press-activate the disinfecting cap 200 to release the disinfecting fluid. For example, the user may grip the gripping element 212 of the housing 202 with the index and middle fingers of their hand and press the pressing surface 264 of the slidable plunger 250, for example with their thumb, to activate the disinfecting cap 200. In certain embodiments, the pressing surface 264 may include one or more longitudinal ribs to provide a uniform press surface. Other configurations of the pressing surface 264 include circular ribs or flat surfaces.

In some embodiments, the pressing surface 264 may be configured to permit rotation of the slidable plunger 250 about the longitudinal axis L (shown in FIG. 6B). According to this embodiment, the gripping features on the pressing surface 264 may be in the form of one or more flattened radially extending wings that may be gripped with the user's other hand to assist the rotational activation of the disinfecting cap 200. In such embodiments, the slidable plunger 250 may be in a threaded engagement with the inner flange 254 such that rotational movement of the slidable plunger 250 about the longitudinal axis L axially moves the slidable plunger 250 toward the inner flange 254. Activation of the disinfecting cap 200 thus involves rotating the slidable plunger 250 clockwise or counterclockwise to threadably move the slidable plunger 250 in the proximal direction to the activated state, thus pressurizing and forcing the disinfecting fluid through the one or more fluid passageways 255.

When the slidable plunger 250 is connected to the open distal end 252 of the housing 202, a proximal end 266 of the slidable plunger 250 may be configured to define an interior volume 210 between the inner flange 254 and the proximal end 266 of the slidable plunger 266 (FIG. 6A). The interior volume 210 of the disinfecting cap 200 may be configured for containing the disinfecting fluid therein. In certain embodiments, the disinfecting fluid may be at least partially absorbed into the compressible absorbent material 214 retained in the interior volume 210, such that compression of the compressible absorbent material 214 releases at least a portion of the disinfecting fluid during activation of the disinfecting cap 200. Alternatively, the interior volume 210 may just contain primarily the disinfecting fluid within the volume such movement of the slidable plunger 250 toward the proximal end 204 of the housing 202 forces the disinfecting fluid through the one or more passageways 256 in the inner flange 254.

The slidable plunger 250 may be held within the housing 202 by a taper fit, a friction fit, or one or more protrusions 268 on a perimeter surface of the slidable plunger 250 or the inner surface 228 of the housing 202 so that the slidable plunger 250 remains stationary unless a predetermined force is applied to the pressing surface 264 to overcome the frictional force of the one or more protrusions 268. In certain embodiments, sliding of the slidable plunger 250 may by prevented unless a minimum force is applied to the pressing surface 264, such as by one or more protrusions 268 or flexible tabs extending radially inward from an inner surface of the open distal end 252 of the housing 202. Activation of the disinfecting cap 200 would then require sufficient force application to the slidable plunger 250 to move the slidable plunger 250 past or over the one or more protrusions 268 or flexible tabs. The slidable plunger 250 is slidable between a first, non-activated state, where the slidable plunger 250 is located at the open distal end 252 of the housing 202 and defines the interior volume 210, and a second, activated state, where the slidable plunger 250 is moved axially in a proximal direction by applying pressure to the pressing surface 264. As the slidable plunger 250 is moved to the activated state, the proximal end 266 of the slidable plunger 250 compresses the compressible absorbent material 214 and/or the disinfecting fluid in the interior volume 210, creating an increase in pressure of the disinfecting fluid in the interior volume 210. Once the pressure of the disinfecting fluid in the interior volume 210 reaches a threshold pressure, the disinfecting liquid is forced through the one or more fluid passageways 256 in the inner flange 254 and contacts one or more surfaces of the fluid path element 106, thereby disinfecting those surfaces. Other embodiments may include one-way valves extending through inner flange 254 so that disinfecting fluid flows through the one-way valves to contact fluid path element 106.

After activation, the disinfecting cap 200 is left engaged with the fluid path element 106 in the activated state for a predetermined length of time sufficient to disinfect all outer surfaces from any microbial or pathogenic materials. The disinfecting cap 200 may also act as a dust cap, preventing contamination of the distal end of the fluid path element 106 with dust particles that may be floating in the air in the injection suite. The disinfecting cap 200 may be left in place during until the next patient and the injector are prepared for the subsequent patient's injection procedure, at which they can be removed and the disinfected/sterilized distal end of the multi-patient fluid path element 106 may be engaged with the proximal end of the single-patient fluid path element 108.

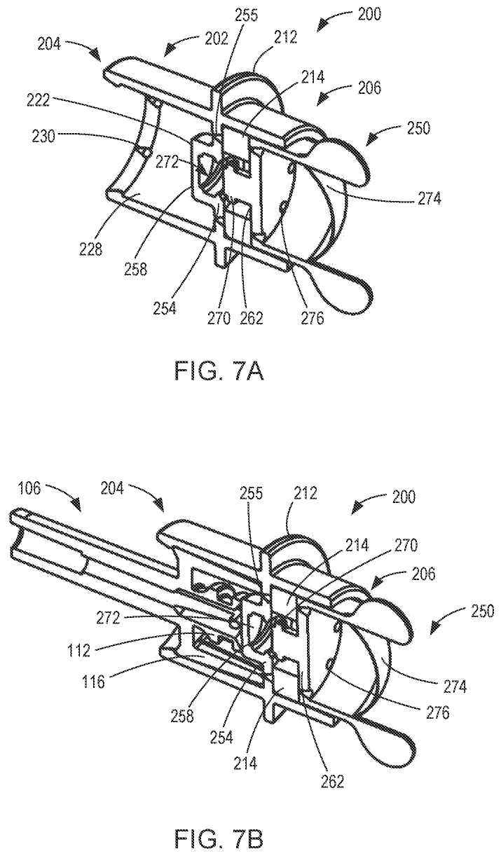

With reference to FIGS. 7A-7B, the slidable plunger 250 and the inner flange 254 are threadably connected via a threaded arrangement 270. In some embodiments, the threaded arrangement 270 has corresponding threaded surfaces that, when rotated, slide the slidable plunger 250 axially between the non-activated and activated states. The slidable plunger 250 may include a fluid reservoir 272 for containing a volume of a disinfecting fluid. A distal wall 274 of the slidable plunger 250 is made of a pliable, deformable elastomeric material. In certain embodiments, the fluid reservoir 272 may also include a compressible absorbent material at least partially saturated with the disinfecting fluid.

With continued reference to FIGS. 7A-7B, the proximal end 262 of the slidable plunger 250 has one or more pressure-activated passageways 276, such that disinfecting fluid may flow through the pressure-activated passageways 276 when pressurized. In this embodiment, the disinfecting cap 200 need not include the compressible absorbent material and/or the disinfecting fluid in the interior volume 210 defined between the slidable plunger 250 and the inner flange 254. Instead, upon rotatable movement of the slidable plunger 250 from the non-activated stated to the activated state, the user can apply pressure to the distal wall 274 of the slidable plunger 250, either during rotation or after the slidable plunger 250 has rotatably contacted the inner flange 254. The disinfecting fluid in the fluid reservoir 272 is thus pressurized and travels through the pressure-activated passageways 276 and through the one or more passageways 255 in the inner flange 254 to contact and disinfect any exterior surface of the fluid path element 106 (shown in FIG. 7B). In some embodiments, the pressure-activated passageways 276 may be in a normally closed position prior to activation of the slidable plunger 250 by pushing the distal wall 274 in a proximal direction. The disinfecting cap 200 may include a fluid path sealing surface 222, as described herein, that creates a fluid tight seal against the lumen 112 of the fluid path element 106 and prevents any disinfecting fluid or other contaminant from entering the lumen 112.

In certain embodiments, the fluid reservoir 272 may include a thin membrane over the pressure-activated passageways 276 to retain the disinfecting fluid. Upon application of sufficient pressure to the distal wall 274 in the proximal direction, disinfecting fluid in the fluid reservoir 272 is pressurized to a pressure sufficient to break the thin membrane and allow the disinfecting fluid to exit the fluid reservoir 272.

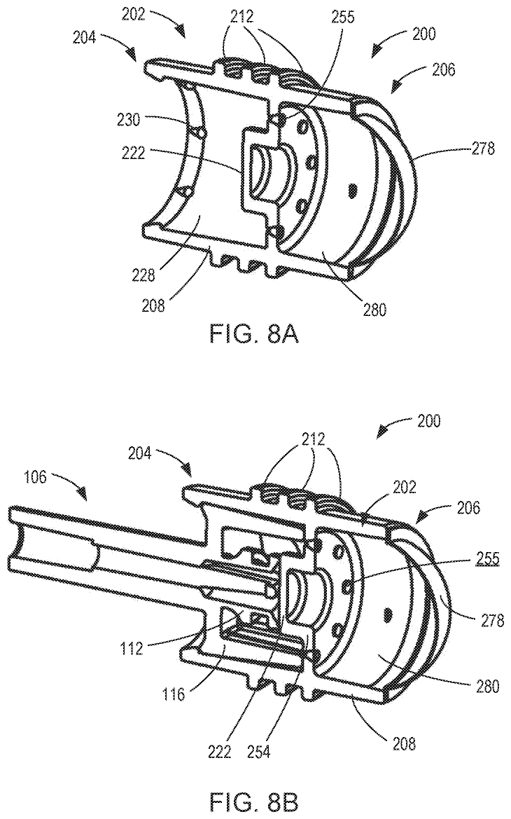

With reference to FIGS. 8A-8B, the disinfecting cap 200 has a housing 202 including an open proximal end 204 configured to releasably engage an outer surface of the fluid path element 106, a closed distal end 206 which is enclosed by a pliable, deformable elastomeric membrane 278, and a sidewall 208 extending between the open proximal end 204 and the closed distal end 206. The disinfecting cap 200 further includes an inner fluid reservoir 280 defined by the pliable, deformable elastomeric membrane 280, the inner surfaces 228 of a distal portion of the sidewall 208, and an inner flange 254 extending radially inward from the sidewall 208 to define the inner fluid reservoir 280. The inner fluid reservoir 280 may be filled with a disinfecting fluid. In certain embodiments, the inner fluid reservoir 280 may also include a compressible absorbent material, such as the compressible absorbent material described herein with reference to FIGS. 2A-2F, at least partially saturated with the disinfecting fluid. In certain embodiments, the compressible absorbent material, for example a sponge, may act as a spring to keep the pliable, deformable elastomeric membrane 278 in the distended state when no pressure is applied and release the disinfecting fluid when pressure is applied to the pliable, deformable elastomeric membrane 278 by the user. After the user releases the pressure on the elastomeric membrane 278, the compressible absorbent material may return to its original, non-compressed state. As the compressible absorbent material returns to the non-compressed state, it may absorb excess disinfecting fluid and cause elastomeric membrane 278 to return to the extended state, upon which time the elastomeric material may be re-compressed a second time.

The inner flange 254 may include a fluid path sealing surface 222 (shown in FIG. 8A) on its proximal side. The fluid path sealing surface 222 may be configured for sealing the lumen 112 of the fluid path element 106 (shown in FIG. 8B) to prevent ingress of any of the disinfecting fluid into an interior volume of the lumen 106 when the disinfecting cap 200 is activated or to prevent dripping of any fluid from the interior of the lumen 112. The inner flange 254 may further include one or more fluid passageways 255 as described herein that allow fluid communication between the inner fluid reservoir 280 and the proximal interior volume 210 of the disinfecting cap 200.

Once engaged with the fluid path element 106, for example after removal of the used single patient fluid path element, the disinfecting cap 200 may be activated by applying pressure to the pliable, deformable elastomeric membrane 278 in a proximal direction. For example, the user may apply pressure with a thumb or other digit by pressing on the elastomeric membrane 278. Once the disinfecting fluid within the reaches a predetermined pressure, the one or more fluid passageways 276 may open such that the disinfecting fluid flows through the fluid passageways 255 and contacts one or more surfaces of the fluid path element 106. After activation, the disinfecting cap 200 may be left engaged with the fluid path element 106 in the activated state for a time sufficient to disinfect outer surfaces of the fluid path element 106 from any microbial or pathogenic materials.

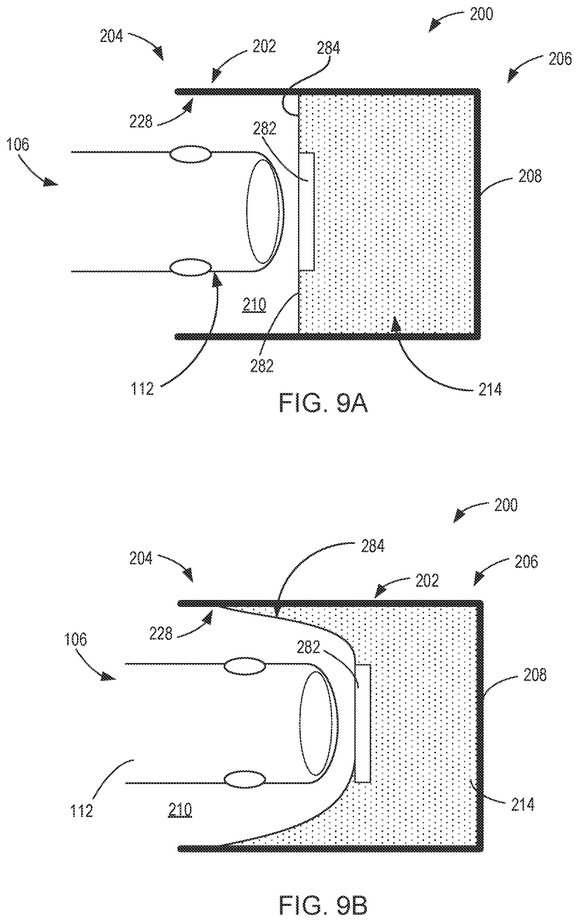

With reference to FIGS. 9A-9B, the disinfecting cap 200 may include a housing 202 having an open proximal end 204, a closed distal end 206 and a sidewall 208 extending between the open proximal end 204 and the closed distal end 206. The disinfecting cap 200 may further include a sealing member 282 attached to the inner surface 228 of the sidewall 208. The sealing member 282 may be configured to fluidly seal the inner volume of the lumen 112 of the fluid path element 106 when the fluid path element 106 is connected to the disinfecting cap 200 to prevent ingress of contaminants or egress of fluid therefrom. Extending radially outward from the sealing member 282 toward the inner surface 228 of the sidewall 208 is a thin deflectable material 284. The deflectable material 284, in its non-deflected state, along with the sealing member 282 and the closed distal end 206 define a proximal end of the interior volume 210 of the disinfecting cap 200.

The proximal end of the interior volume 210 may include a compressible absorbent material 214 therein which is at least partially saturated with a disinfecting fluid. The disinfecting cap may be moved from a first, non-activated state to a second, activated state by pressing on the distal end of the disinfecting cap 200 or otherwise moving the disinfecting cap 200 in a proximal direction. As the disinfecting cap 200 is activated, such as by pushing the fluid path element 106 toward the distal end 206 of the disinfecting cap 200, the distal end of lumen 112 of the fluid path element 106 pushes against the sealing member 282, thereby sealing the inner portions of lumen 112, and deflecting the thin deflectable material 284. This movement decreases the volume of the interior fluid volume and compresses the compressible absorbent material 214 and the disinfecting fluid contained therein. The pressurized fluid then presses against the thin deflectable material 284, deflecting the material in a proximal direction and allowing the disinfecting fluid to flow past the thin deflectable material and contact one or more exterior surfaces of the fluid path element 106. Contact of the disinfecting fluid with the various surfaces of the fluid path element 106 for a predetermined length of time, such as from several second up to 7 days, effectively disinfects the contacted surfaces.

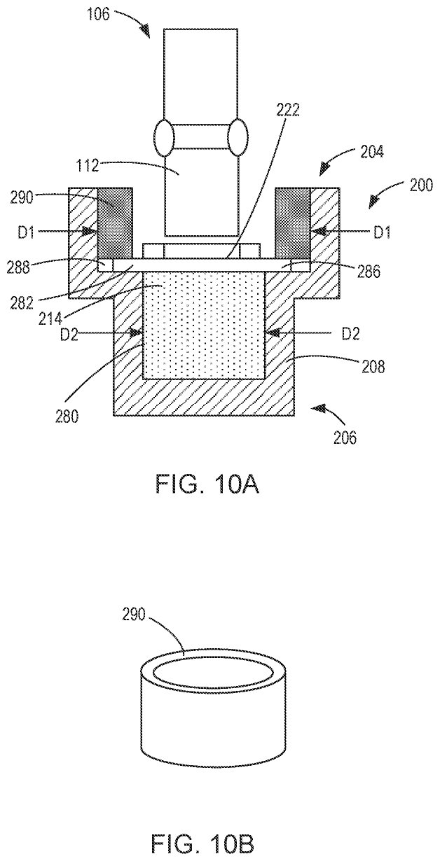

With reference to FIG. 10A, the disinfecting cap 200 may include a proximal end 204 having a larger inner diameter D1 relative to an inner diameter D2 of the closed distal end 206. As described herein, the distal end 206 may include an inner fluid reservoir 280 defined by the closed distal end 206, the distal portion of the sidewall 208, and the sealing member 282. The inner fluid reservoir 280 may be filled with a disinfecting fluid or an absorbent material 214 at least partially saturated with a disinfecting fluid, as described herein.

The sealing member 282 may include a flexible flange 286 extending around at least a portion of the circumference of the fluid path sealing portion 222. The sealing member 280 may be sealably seated against a lip 288 formed at the transition from the larger inner diameter D1 and the smaller inner diameter D2, thus preventing release of the disinfecting fluid from the inner fluid reservoir 280 when the disinfecting cap 280 is in the non-activated state. As described herein, pressure of the lumen 112 of the fluid path element 106 on the proximal sealing surface of the sealing member 282 may create a fluid tight seal between the interior of the lumen 112 and the fluid path sealing surface 222. Further pressure on the fluid path element 106 against the sealing member 282 in the distal direction may break the seal between the sealing member 282 and the lip 288 and push the sealing member 282, with flexing of the flexible flange 286, into the interior volume of the distal end 206. With concomitant pressure increase on the disinfecting fluid or on the compressible absorbent material at least partially saturated with the disinfecting fluid, the disinfecting fluid is released toward the fluid path element 106 around the periphery of the flexed flexible flange 286, coating one or more outer surfaces of the lumen 112 and fluid path element 106 surfaces and sterilizing the end of the fluid path element 106. In certain embodiments, the disinfecting cap 200 may further include a ring 290 of an absorbent material arranged around at least a portion of the inner periphery of the larger diameter proximal section of the disinfecting cap 200. The ring 290 of absorbent material (see FIG. 10B) may help retain the seal between sealing member 282 and lip 288 and may be in contact with an outer surface of the fluid path element 106 absorb excess disinfecting fluid and ensuring fluid path member 106 is completely coated with disinfecting fluid.

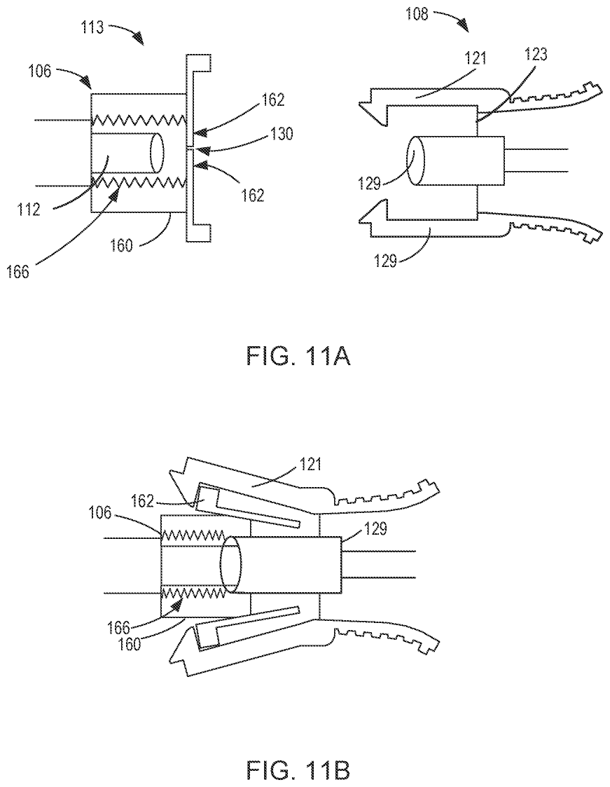

With reference to FIGS. 11A-11B, the distal end 113 of the fluid path element 106 may include features that prevent microbial or pathogenic contamination of the lumen 112 of the fluid path element 106 by one or more movable elements. For example, in a first embodiment, the fluid path element 106 may include a cover 130 to prevent contamination of the lumen 112 and prevent fluid dripping from the end of the fluid path element 106. The cover 130 may include a shroud 160 with movable protecting abutment 162 and a compressible side cover 166. As illustrated in FIGS. 11A-11B, the distal end 113 of the fluid path element 106 has the shroud 160 that surrounds the fluid path element 106. A distal end of the shroud 160 has a movable two-piece structure having the sealed protecting abutment 162 that is positioned over the lumen 112. The single-patient fluid path set 108 may have a pair of flexible legs 121 that are configured to pivot radially outward about a pivot point 123. As the ends of the extending flexible legs 121 of the single-patient fluid path set 108 contact the distal surface of the shroud 160, the sealed protecting abutment 162 is moved from a covering position (FIG. 11A) to an uncovered position (FIG. 11B) as the flexible legs 121 push against the sealed protecting abutment 162. The compressible side cover 166 is then compressed from an uncompressed position (FIG. 11A) to a compressed position (FIG. 11B) as a lumen 129 of the single-patient fluid path set 108 engages the lumen 106 of the fluid path element 106.

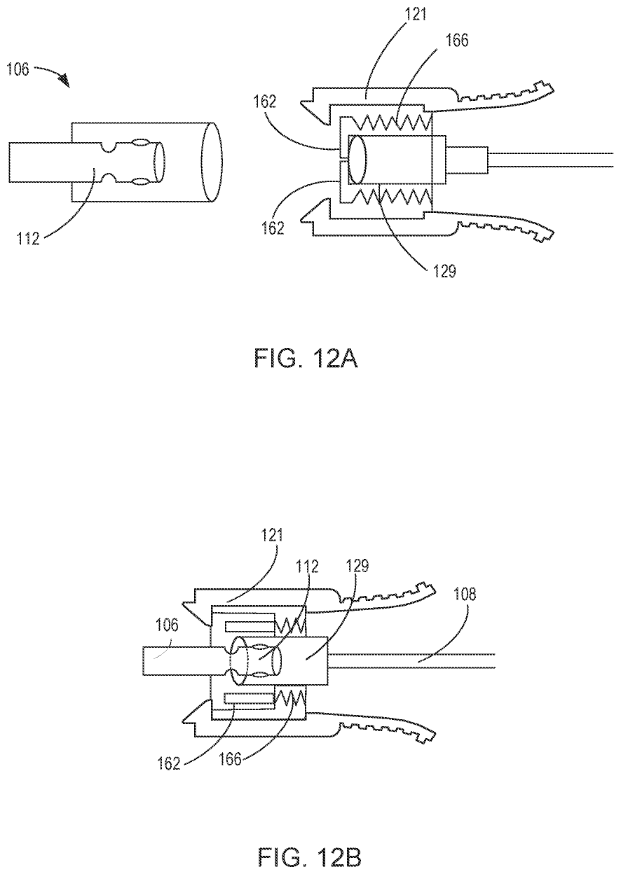

With reference to FIGS. 12A-12B, the cover 130 may be provided on the distal end of the single-patient fluid path set 108. For example, the compressible side cover 166 may extend around the lumen 129 of the single-patient fluid path set 108 and may be configured to be compressed in a proximal direction as the lumen 112 of the fluid path element 106 contacts the sealed protecting abutment 162, thereby causing the two-piece structure to transition from the covered position (FIG. 12A) to the uncovered position (FIG. 12B). As the lumen 112 of the fluid path element 106 is engaged with the lumen 129 of the single-patient fluid path set 108, the compressible side cover 166 is compressed from an uncompressed position (FIG. 12A) to a compressed position (FIG. 12B).

As the single patient fluid path set 108 is engaged with the multi-patient fluid path element 106, the single-patient fluid path set 108 moves the flexible engagement legs 121 in a radially outward direction. As the engagement legs 121 flex outward, the silicone sealing members are moved radially apart allowing access to the lumen 106 of the multi-patient fluid path element 106 so the single patient fluid path set 108 may be engaged therewith.

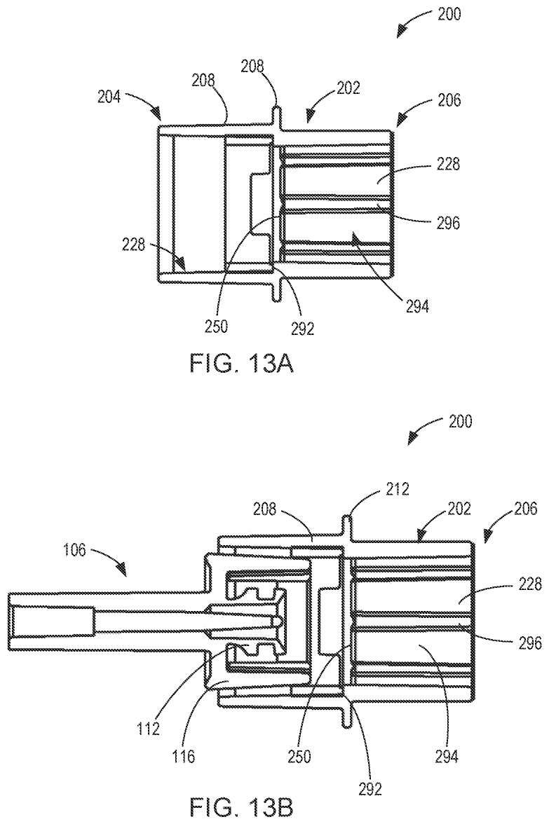

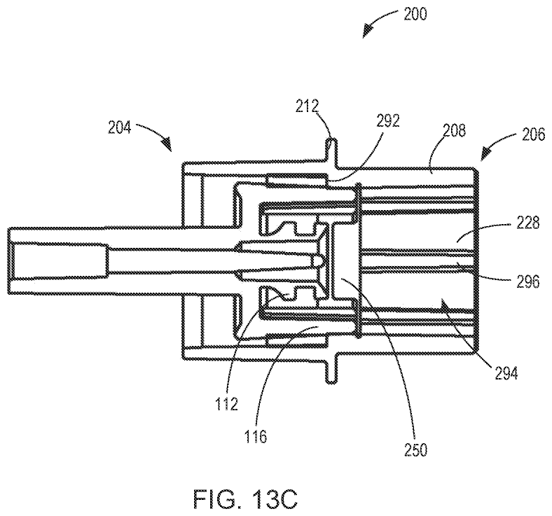

With reference to FIGS. 13A-13C, the housing 202 of the disinfecting cap 200 may include a sealing lip 292 extending radially inward from the inner surface 228 of the sidewall 208. The sealing lip 292 is positioned between the open proximal end 204 and the closed distal end 206 within the interior volume 210 of the housing 202. A slidable plunger 250 may be sealably seated on a proximal side of the sealing lip 292 so that a distal fluid volume 294 is defined by the distal portion of the sidewall 208, the slidable plunger 250, and the closed distal end 206. The inner surface 228 of the sidewall 208 may include a plurality of longitudinal grooves or protrusions 296 that define fluid passageways for the disinfecting fluid to flow through. In some embodiments, the plurality of longitudinal grooves or protrusions 296 may be spaced apart at equal or unequal angular intervals. The distal fluid volume 294 may include a compressible absorbent material, such as the compressible absorbent material 214 described herein with reference to FIGS. 2A-2F, at least partially saturated with a disinfecting fluid. Alternatively, the distal fluid volume 294 may simply contain a volume of the disinfecting fluid sealably retained within the distal fluid volume 294 by the slidable plunger 250.

As the disinfecting cap 200 is releasably engaged with the distal end of the fluid path element 106, the fluid path element 106 contacts the slidable plunger 250 in a first, non-activated state. In certain embodiments, the slidable plunger 250 may form a fluid tight seal against a distal end of the lumen 112 of the fluid path element 106, as described herein, thereby sealing the lumen 112 and preventing contamination of the lumen 112 and/or dripping of fluid out of the lumen 112. Moving the disinfecting cap 200 from the first, non-activated state to the second, activated state comprises pressing the disinfecting cap 200 further onto the fluid path element 106 such that the fluid path element 106 is further inserted into the interior volume 210 of the housing 202 toward the closed distal end 206. Additional movement of the disinfecting cap 200 relative to the fluid path element 106 may push the slidable plunger 250 past the sealing lip 292, thereby reducing the volume of the distal fluid volume 294 and pressurizing the disinfecting fluid contained therein.

As the slidable plunger 250 is pushed via movement of the fluid path element 106 past the proximal ends of the plurality of longitudinal grooves or longitudinal protrusions 296 (FIG. 13C), the disinfecting fluid can flow through the plurality of longitudinal grooves or around the plurality of longitudinal protrusions 296 past the slidable plunger such that the disinfecting fluid can contact outer surfaces of the fluid path element 106. Contact of the disinfecting fluid with various surfaces of fluid path element 106 for a predetermined length of time, such as from several second up to 7 days, effectively disinfects the contacted surfaces.

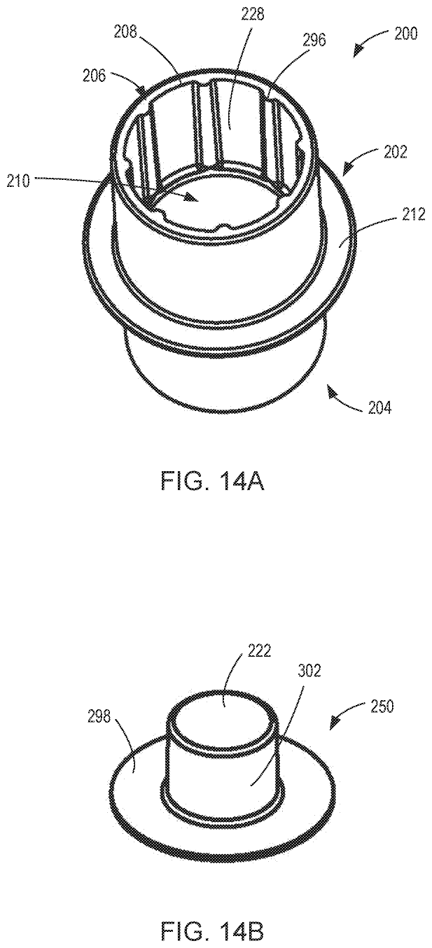

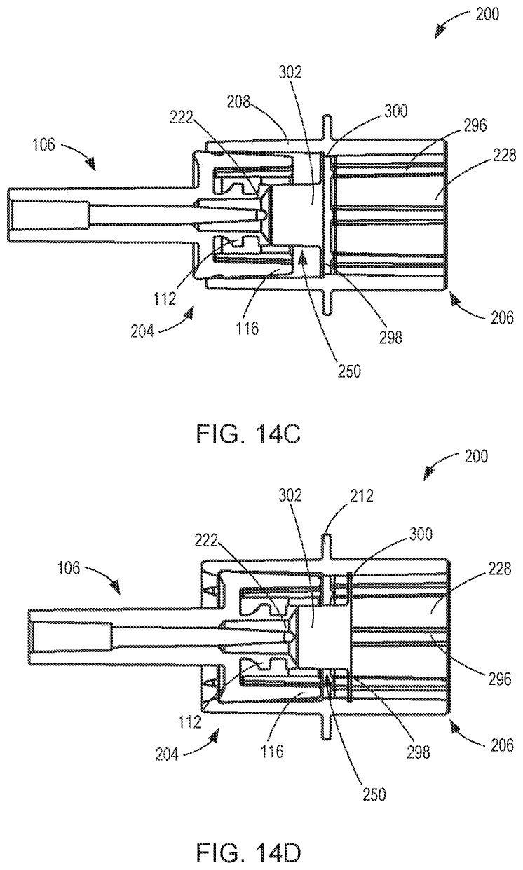

With reference to FIGS. 14A-14D, the disinfecting cap 200 may have a slidable plunger 250 (shown in FIGS. 14B-14D) having an inner sealing member 298 that forms a circumferential seal with an inner surface sealing seat 300 defined by the inner surface 228 of the housing 202. As the disinfecting cap 200 is pushed toward the fluid path element 106 to an activated position, the inner sealing member 298 is moved out of contact with the inner surface sealing seat 300 and toward the closed distal end 206 of the housing 202. Similar to the embodiment discussed herein with reference to FIGS. 13A-13C, the inner surface 228 of the sidewall 208 distal of the inner surface sealing seat 300 may include a plurality of longitudinal grooves or protrusions 296 that define fluid passageways for the disinfecting fluid to flow through. Once the slidable plunger 250 is pushed distally away from the inner surface sealing seat 300, the disinfecting fluid contained in distal fluid volume 294 can flow in the proximal direction toward fluid path element 106 through the plurality of longitudinal grooves or protrusions 296.

As best shown in FIG. 14B, the slidable plunger 250 has an extending feature 302 that defines the fluid path sealing surface 222 that is configured to seal the lumen 112 of the fluid path element 106 in both the inactivated position, when the lumen 112 first contacts the slidable plunger 250, and the activated position, when the fluid path element 106 pushes the slidable plunger 250 distally toward the closed distal end 206.

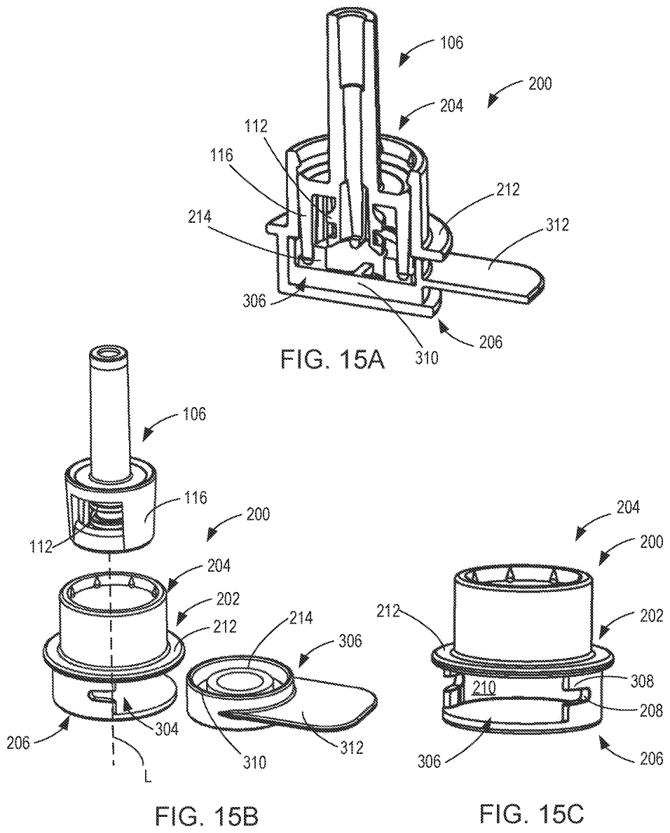

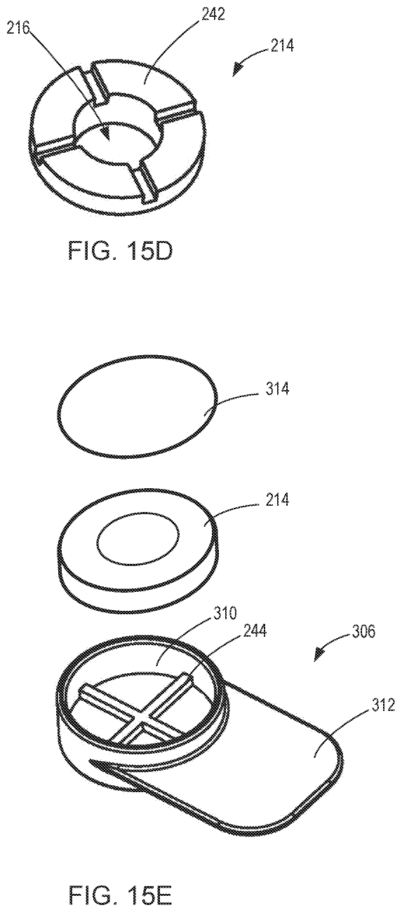

With reference to FIGS. 15A-15E, the disinfecting cap 200 is shown in accordance with another embodiment of the present disclosure. As best shown in FIG. 15C, the housing 202 of the disinfecting cap 200 has a lateral opening 304 between the open proximal end 204 and the closed distal end 206. The lateral opening 304 extends through the sidewall 208 of the housing 202 into the interior volume 210 of the housing 202. In some embodiments, the lateral opening 304 may be configured to receive an insertable tray 306 (shown in FIGS. 15B and 15E) containing the compressible absorbent material 214. The lateral opening 304 has at least one lateral groove 308 configured to receive at least a portion of the insertable tray 306 that allows swiveling movement of the tray 106 relative to the housing 202 to provide a scrubbing action between the compressible absorbent material 214 and the fluid path element 106 when the tray 106 is inserted into the housing 202 and in contact with a surface of the fluid path element 106.

With reference to FIG. 15E, the insertable tray 306 has a receiving portion 310 configured to receive the compressible absorbent material 214 at least partially saturated with a disinfecting material (shown in FIG. 15D) and a handling tab 312 extending radially outward from the receiving portion 310. The receiving portion 310 may be sized to be received within the lateral opening 304, while the handling tab 312 may be configured to be received within the lateral groove 308 of the lateral opening 304 during a swiveling action. The insertable tray 306 may be inserted into the interior volume 210 of the housing 202 through the lateral opening 304 by sliding the insertable tray 306 in a direction perpendicular to the longitudinal axis L (shown in FIG. 15B). Once inserted, the insertable tray 306 may be swiveled or rotated reciprocally in a direction about the longitudinal axis L by a user grasping a handling tab 312 to provide a scrubbing action between the compressible absorbent material 214 and of the fluid path element 106. The compressible absorbent material 214 and the insertable tray 306 may have corresponding anti-rotation slot 242 and anti-rotation protrusions 244, as described herein, for preventing a rotation of compressible absorbent material 214 relative to insertable tray 306.

As shown in FIG. 15E, the insertable tray 306 may be provided with a removable seal 314 to seal the compressible absorbent material 214 within the tray 306 and retain the disinfecting fluid. The removable seal 314 may be removed prior to insertion of the insertable tray 306 into the disinfecting cap 200. In another embodiment, the insertable tray 306 and compressible absorbent material 214 may be provided without the disinfecting fluid and the removable seal 314 may be provided to retain the sterility of the interior volume of the insertable tray 306. Prior to insertion into the disinfecting cap 200, the removable seal 314 may be removed and the compressible absorbent material 214 may be saturated with the disinfecting fluid from a bulk fluid source. In certain embodiments, the compressible absorbent material 214 may be provided in a form defining the central opening 216 that substantially corresponds to the outer diameter of the lumen 112 of the fluid path element 106. During contact of the compressible absorbent material 214 with the fluid path element 106, the lumen 112 of the fluid path element 106 may not contact the compressible absorbent material 214. In other embodiments, the compressible absorbent material 214 may not include central opening 216.

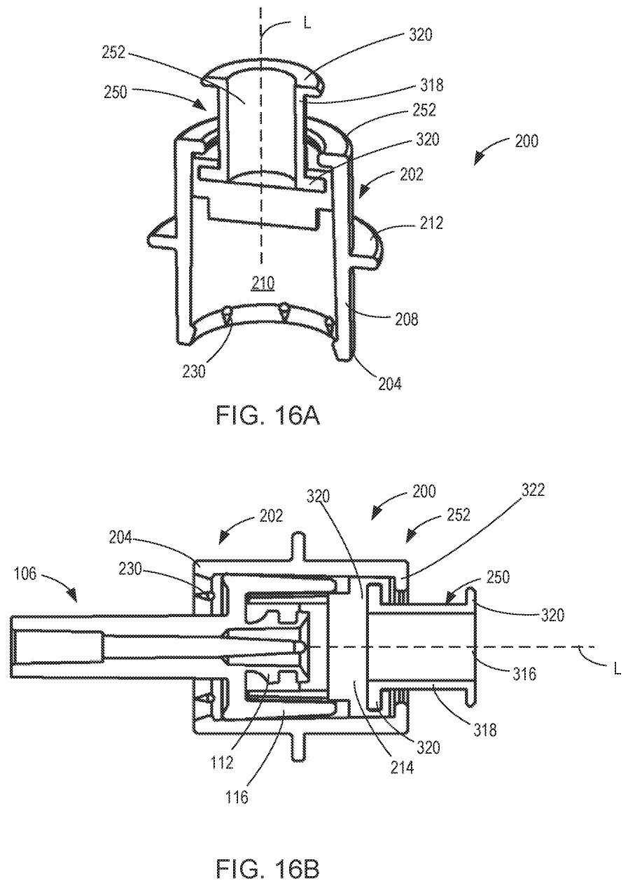

With reference to FIGS. 16A-16B, the disinfecting cap 200 may include the slidable plunger 250 having an internal chamber 316 configured to contain a volume of the disinfecting fluid. The slidable plunger 250 may be configured for reciprocal movement in a direction along the longitudinal axis L within the interior volume 210 of the housing 202. The slidable plunger 250 has a cylindrical body 318 with a pair of retaining flanges 320 that delimit the travel of the slidable plunger 250 relative to the housing 202 by interacting with a distal flange 322 at the open distal end 252 of the housing 202. In some embodiments, the slidable plunger 250 may further be configured for rotational movement about the longitudinal axis L.

With continued reference to FIGS. 16A-16B, a proximal end 324 of the slidable plunger 250 is connected to the compressible absorbent material 214. For example, the compressible absorbent material 214 may have a groove 326 configured to receive a proximal retaining flange 320 of the slidable plunger 250. In this manner, the compressible absorbent material 214 is movable with movement of the slidable plunger 250. Once the disinfecting cap 200 is connected to the fluid path element 106, the internal volume 316 of the slidable plunger 250 may be filled with the disinfecting fluid such that the disinfecting fluid at least partially saturates the compressible absorbent material 214. As the slidable plunger 250 is axially moved towards and away from the fluid path element 106 in the direction along the longitudinal axis L, such as by a user gripping the distal retaining flange 320 and gripping element 212, the compressible absorbent material 214 contacts the fluid path element 106 to sterilize the fluid path element 106. In some embodiments, the slidable plunger 250 further may be rotated about the longitudinal axis L to scrub the fluid path element 106.

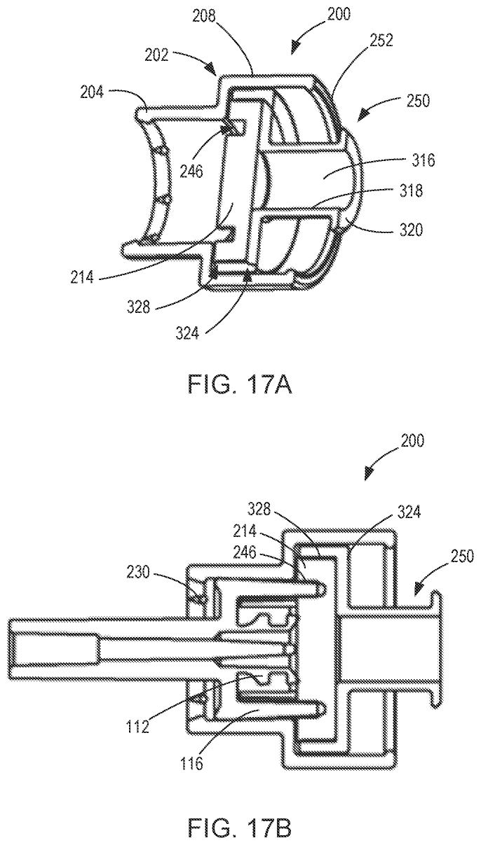

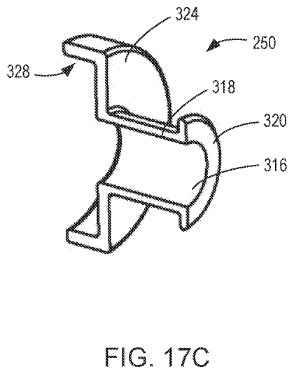

With reference to FIGS. 17A-17C, the disinfecting cap 200 is shown in accordance with another embodiment that is a variation of the embodiment shown in FIGS. 16A-16B. Instead of the cylindrical body 318 having a uniform diameter throughout its length, as shown in FIGS. 16A-16B, the proximal end 324 of the slidable plunger 250 may have a wider diameter compared to a diameter of the cylindrical body 318. In some embodiments, the proximal end 324 may define a receiving cavity 328 for receiving the compressible absorbent material 214 therein. As described herein, the compressible absorbent material 214 may include a circumferential groove 246 for interfacing with the outer skirt 116 of the fluid path element 106. The wider diameter of the portion of sidewall 208 of disinfecting cap 200 may further act as gripping element 212 when compressing the slidable plunger 250.

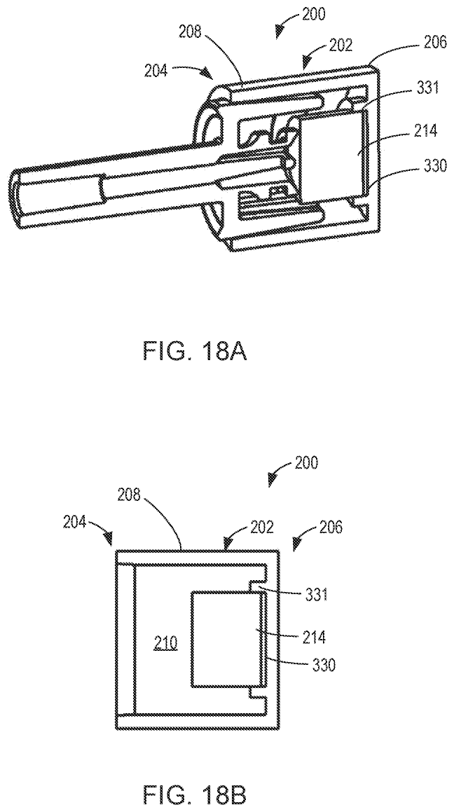

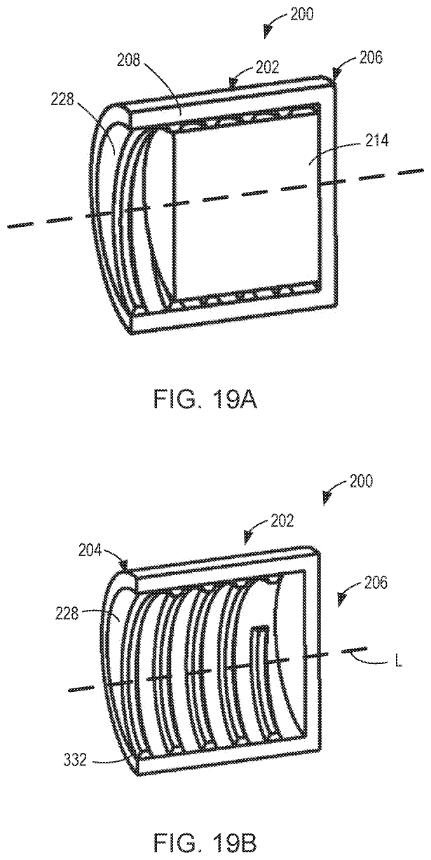

With reference to FIGS. 18A-18B, the disinfecting cap 200 may include a housing 202 having an open proximal end 204 and a closed distal end 206 with a sidewall 208 extending between the open proximal end 204 and the closed distal end 206 defining an interior volume 210 (shown in FIG. 18B). The compressible absorbent material 214 is retained within the interior volume 210 and connected to the closed distal end 206 via an adhesive pad 330. In some embodiments, the adhesive pad 330 may include a low temperature melt polymer. The proximal end of the compressible absorbent material 214 may include a fluid impervious coating configured to sealably engage the lumen 112 of fluid path element 106 to prevent ingress of disinfecting fluid into the fluid path of lumen 112. For example, the fluid impervious coating may include a layer of close celled polymer, a silicone layer, a polymer layer or other suitable material adhered to the proximal end of the compressible absorbent material 214. The closed distal end 206 may have a retainer 331, such as a circumferential wall protruding proximally from the closed distal end 206, for retaining compressible absorbent material 214.

With reference to FIGS. 19A-19B, the disinfecting cap 200 is shown in accordance with another embodiment of the present disclosure. The inner surface 228 of the housing 202 may have one or more protrusions 332 that protrude radially inward from the inner surface 228. The one or more protrusions 332 may extend along at least a portion of a longitudinal length of the housing 202 in the direction of the longitudinal axis L. In some embodiments, the one or more protrusions 332 maybe configured as a helical thread that is configured to threadably interface a corresponding thread on the compressible absorbent material 214. In other embodiments, the one or more protrusions 332 may be configured to compressively retain the compressible absorbent material 214, such as due to an interference fit. The proximal end of the compressible absorbent material 214 may include a fluid impervious coating configured to sealably engage the lumen 112 of fluid path element 106 to prevent ingress of disinfecting fluid into the fluid path of lumen 112, as described herein.

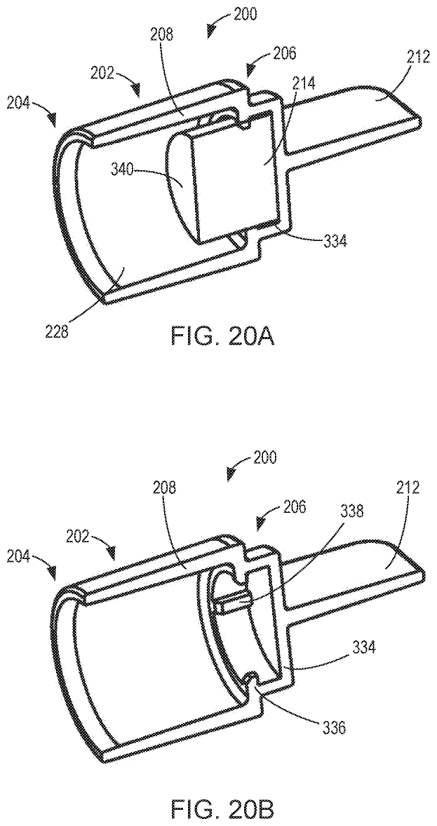

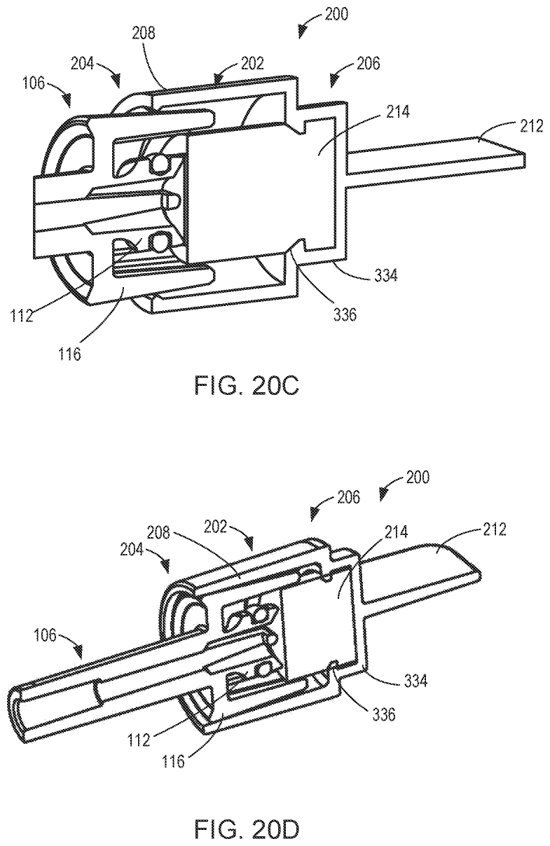

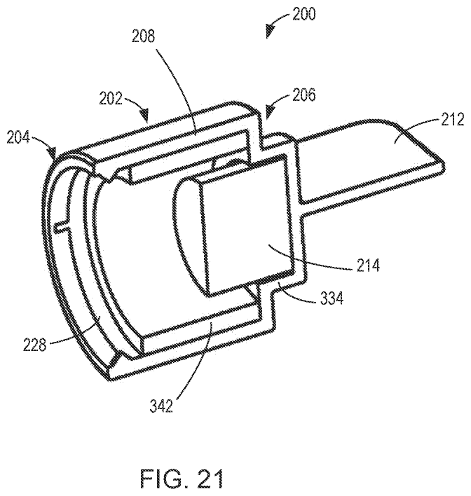

With reference to FIGS. 20A-20D, the disinfecting cap 200 is shown in accordance with another embodiment of the present disclosure. The closed distal end 206 may have a retaining pocket 334 that is configured to retain the compressible absorbent material 214. In some embodiments, the retaining pocket 334 may have a smaller inner diameter compared to a diameter of the inner surface 228 of the housing 202. The inner diameter of the retaining pocket 334 may be substantially identical to an outer diameter of the compressible absorbent material 214. The retaining pocket 334 may have one or more undercut portions 336, for example at the transition from the retaining pocket 334 and the inner surface 228 of the housing 202 or elsewhere in the retaining pocket 334 to engage and hold a distal end of the compressible absorbent material 214 securely in the retaining pocket 334.

In some embodiments, the retaining pocket 334 may include one or more longitudinal ribs 338 extending radially inwardly and configured to engage a side surface of the compressible absorbent material 214. The one or more longitudinal ribs 338 may prevent the compressible absorbent material 214 from rotating when the disinfecting cap 200 is rotated relative to the fluid path element 106 (shown in FIGS. 21C-21D) to create a scrubbing effect between the fluid path element 106 and the compressible absorbent material 214 to affect the disinfecting action of the fluid path element 106. In certain embodiments, a fluid impervious coating or film 340, as described herein, may be attached to the proximal surface of the compressible absorbent material 214 to act as a blocker to prevent the disinfecting fluid from the compressible absorbent material 214 from moving into the lumen 112 of the fluid path element 106. According to these embodiments, the film 340 may be attached or otherwise adhered to the surface of the compressible absorbent material 214, for example by an adhesive, co-molding, or a melt process on the front of the compressible absorbent material 214 to close the open cells of the compressible absorbent material 214 and block fluid flow through that portion of the surface of the compressible absorbent material 214.

With reference to FIG. 21, the disinfecting cap 200 may include an outer cylindrical sleeve 342 surrounding the compressible absorbent material 214. The outer cylindrical sleeve 342 may be made from the same material as the compressible absorbent material 214. In this embodiment, the compressible absorbent material 214 may provide a scrubbing and disinfecting action on the interior portions of the fluid path element 106 (shown in FIG. 1B) while the outer cylindrical sleeve 342 may provide a scrubbing and disinfecting action on the outer portions of the fluid path element 106. The outer cylindrical sleeve 342 may be retained by one or more retention undercuts 336 protruding from the inner surface 228 of the housing 200. The proximal end of the compressible absorbent material 214 may include a fluid impervious coating configured to sealably engage the lumen 112 of fluid path element 106 to prevent ingress of disinfecting fluid into the fluid path of lumen 112, as described herein.

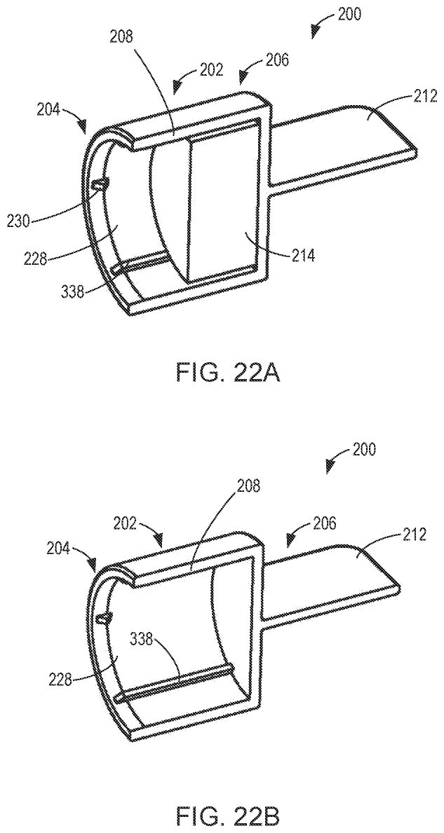

With reference to FIGS. 22A-22B, the disinfecting cap 200 may have a uniform inner diameter between the open proximal end 204 and the closed distal end 206. At least a portion of the inner surface 228 of the housing 202 may have one or more longitudinal ribs 338 protruding radially inwardly from the inner surface 228. In some embodiments, the one or more longitudinal ribs 338 may be configured to engage with the compressible absorbent material 214 to prevent rotation of the compressible absorbent material 214 relative to the housing 202 during a twisting action of the disinfecting cap 200 relative to the fluid path element 106 (shown in FIG. 1B) to affect a scrubbing action on the fluid path element 106. The proximal end of the compressible absorbent material 214 may include a fluid impervious coating configured to sealably engage the lumen 112 of fluid path element 106 to prevent ingress of disinfecting fluid into the fluid path of lumen 112, as described herein.

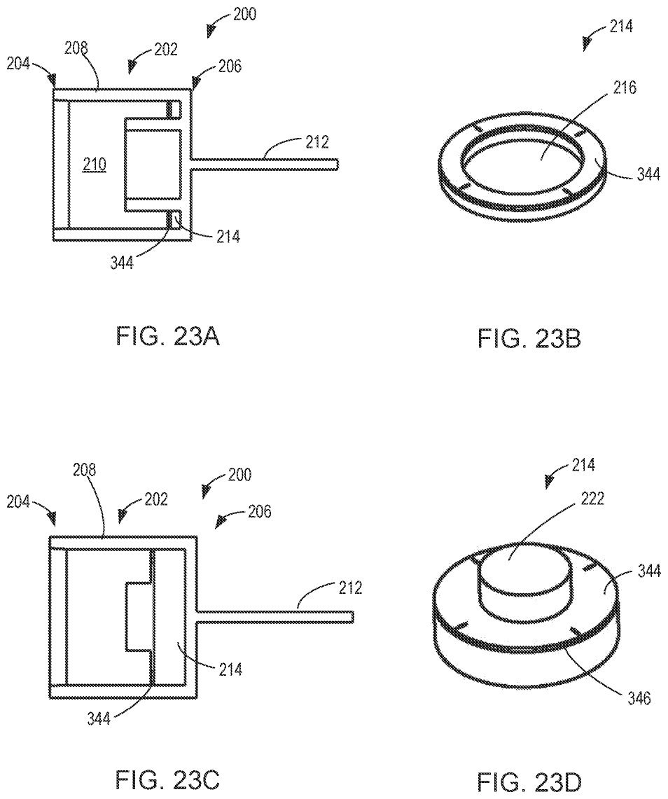

With reference to FIGS. 23A-23D, the disinfecting cap 200 is shown in accordance with other embodiments of the present disclosure. The compressible absorbent material 214 in FIGS. 23A-23B has a central opening 216, while the compressible absorbent material 214 in FIGS. 23C-23D has a protruding fluid path sealing surface 222. The compressible absorbent material 214 in either of the two embodiments may have a fluid impervious coating, film or laminated material 344 adhered or otherwise attached to the proximal surface 248 of the compressible absorbent material 214, as described herein. In some embodiments, the film or laminated material 344 may cover an entirety of the proximal surface 248 such that the film or laminated material 248 extends substantially to the outer circumference of the compressible absorbent material 214. According to certain embodiments, the film or laminated material 344 may include one or more perforations 346 that allow the disinfecting fluid to flow from the compressible absorbent material 214 through the film or laminated material 344 and contact the fluid path element 106 to disinfect a surface of the fluid path element 106.

In other embodiments, a first portion of the film or laminated material 344 may be adhered at a central region of the compressible absorbent material 214 and a remaining portion of the film or laminated material 344 is desirably not adhered to the compressible absorbent material 214. According to these embodiments, as the fluid path element 106 (shown in FIG. 1B) is inserted into the disinfecting cap 200 and compresses the compressible absorbent material 214, the portion of the film or laminate material 344 that is not adhered (i.e., the periphery) is folded away from the compressible absorbent material 214 and around the fluid path element 106. As the film or laminate material 344 folds, a passageway is defined between the fluid path element 106 and the inner surface 228 of the disinfecting cap 200 that allows the disinfecting fluid to flow from the compressible absorbent material 214 toward at least a portion the fluid path element 106 where the disinfecting fluid may contact and disinfect the fluid path element 106.

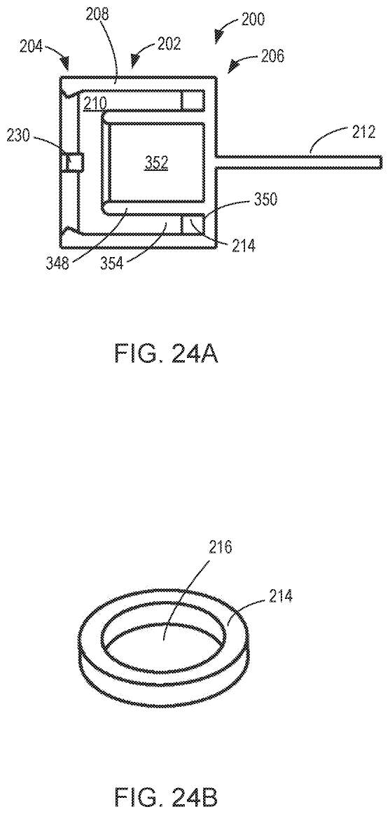

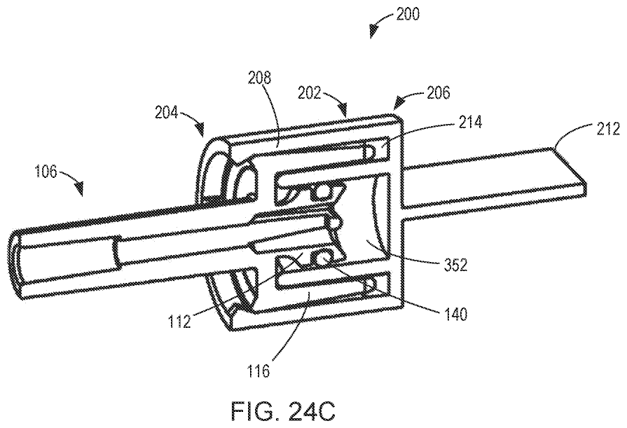

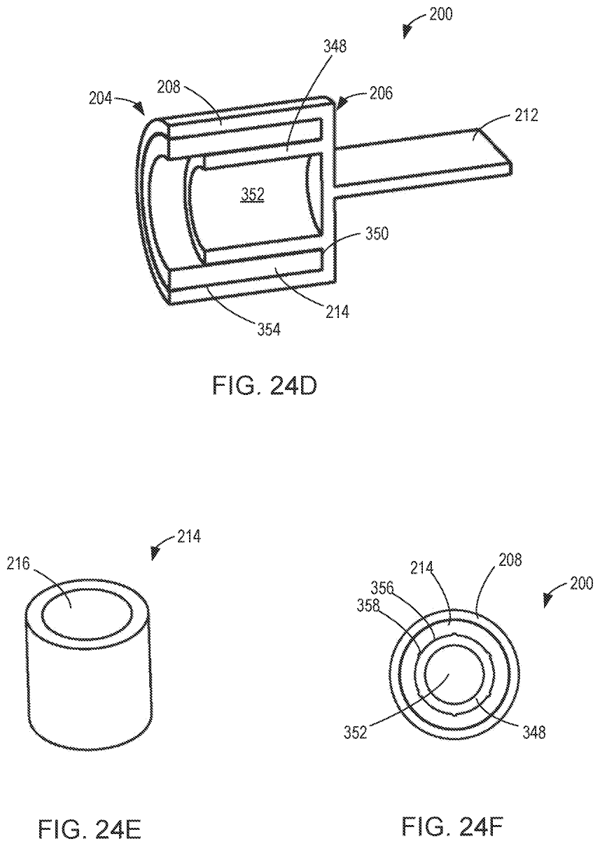

With reference to FIGS. 24A-24C, the disinfecting cap 200 is shown in accordance with another embodiment of the present disclosure. As shown in FIG. 24A, the disinfecting cap 200 may include a housing 202 having an open proximal end 204 and a closed distal end 206 with a sidewall 208 extending between the open proximal end 204 and the closed distal end 206 defining an interior volume 210. The disinfecting cap 200 has an inner sleeve 348 protruding from an inner surface 350 of the closed distal end 206. In some embodiments, the inner sleeve 348 has a cylindrical shape and extends into the interior volume 210 over at least a portion of a longitudinal length of the housing 202. In certain embodiments, the terminal end of the inner sleeve 348 is recessed relative to the open proximal end 204. The inner sleeve 348 defines a receiving space 352 for receiving the lumen 112 of the fluid path element 106 (shown in FIG. 24C). A space 354 between the inner cylindrical sleeve 348 and the inner surface 228 of the sidewall 208 is configured for receiving compressible absorbent material 214. In some embodiments, such as shown in FIG. 24A, the compressible absorbent material 214 may be recessed in the space 354, while in other embodiments, such as shown in FIG. 24D, the compressible absorbent material 214 may protrude proximally from the space 354 relative to the terminal end of the inner sleeve 348. In another embodiment, the compressible absorbent material 214 may terminate distally of the proximal end of the inner sleeve 348. According to this aspect, the disinfecting fluid may be prevented from being squeezed out of the compressible absorbent material 214 before the lumen 112 of the fluid path element 106 sealably enters the receiving space 352, for example by an interface between sealing O-rings on the lumen 112 and the inner side wall of inner sleeve 348, and is shielded by the inner sleeve 348 of the disinfecting cap 200.

In some embodiments, the inner sleeve 348 may be configured to provide a seal with the lumen 112 of the fluid path element 106 to prevent the disinfecting fluid from contacting the lumen 112. In some embodiments, an outer surface of the lumen 112 may have a seal 140 that is configured to contact an inner surface of the inner sleeve 348 to prevent ingress or egress of fluid into and from the lumen 112.

With reference to FIG. 24F, an outer surface 356 of the inner sleeve 348 may have one or more retaining ribs 358 configured to contact at least a portion of the compressible absorbent material 214 to retain the compressible absorbent material 214 in a fixed position relative to the inner sleeve 348 during rotation of the disinfecting cap 200 relative to the fluid path element 106 (shown in FIG. 24C). The one or more retaining ribs 358 may extend axially along a longitudinal length of the inner sleeve 348.

According to another embodiment of the disinfecting cap 200 having the inner sleeve 348, the compressible absorbent material 214 may be retained in the space 354 between the inner sleeve 348 and the sidewall 208, and a proximal end of the compressible absorbent material 214 may be flush with the proximal end of the disinfecting cap to allow disinfection of the fluid path element 106 without moving any microbial material towards the rear portions of the fluid path element 106.

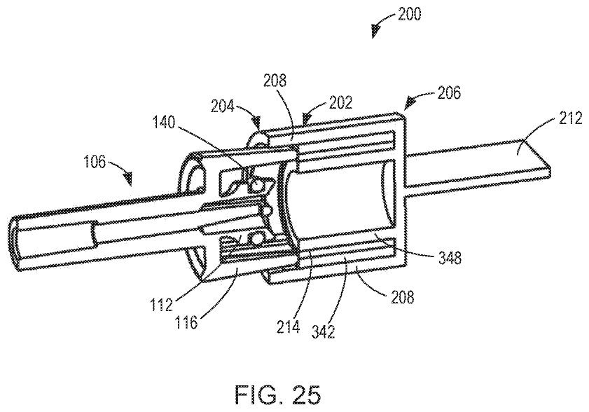

With reference to FIG. 25, the disinfecting cap 200 may include an outer cylindrical sleeve 342 surrounding the compressible absorbent material 214. The outer cylindrical sleeve 342 may be made from the same material as the compressible absorbent material 214 and may be split by a cylindrical cut through substantially along the length of outer cylindrical sleeve 342, wherein the cut is configured to receive the outer skirt 116 of the fluid path element 106. In this embodiment, the compressible absorbent material 214 may provide a scrubbing and disinfecting action on the interior portions of the fluid path element 106 while the outer cylindrical sleeve 342 may provide a scrubbing and disinfecting action on the outer portions of the fluid path element 106. The outer cylindrical sleeve 342 may be retained by one or more retention undercuts protruding from the inner surface 228 of the housing 200. Further, in other embodiments, the split compressible absorbent material 214 may provide rotational scrubbing action as the disinfecting cap 200 is twisted relative to the fluid path element 106. Certain embodiments may include one or more longitudinal ribs, as described herein, to prevent relative movement of the compressible absorbent material 214 and/or the outer cylindrical sleeve 342 during the twisting and scrubbing movement.

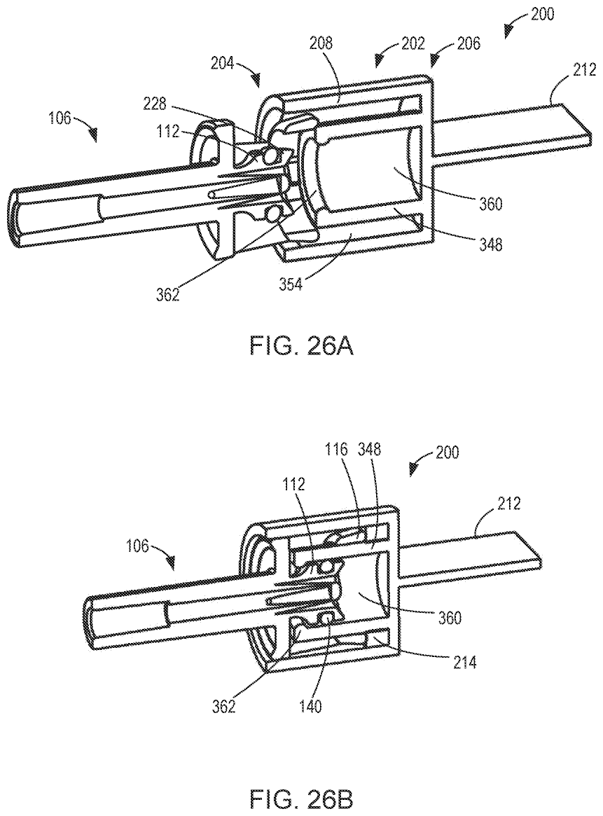

With reference to FIGS. 26A-26B, the disinfecting cap 200 is shown in accordance with another embodiment of the present disclosure. An inner surface 360 of the inner sleeve 348 may include one or more clipping features 362 protruding radially inwardly and configured to releasably engage at least a portion of the lumen 112 of the fluid path element 106. In some embodiments, the one or more clipping features 362 may be configured as a continuous or discontinuous rib that extends around an inner circumference of the inner sleeve 348. The one or more clipping features 362 may retain the disinfecting cap 200 connected to the fluid path element 106 and resist the restoring force from the compressed absorbent material 214 so that the disinfecting cap 200 is retained on the fluid path element 106 for at least an appropriate amount of time for the disinfecting fluid to disinfect any microbial contaminants on the surfaces of the fluid path element 106. In certain embodiments, the one or more clipping features 362 may also provide tactile and/or audible feedback to the user that the disinfecting cap 200 has been appropriately installed on the fluid path element 106.

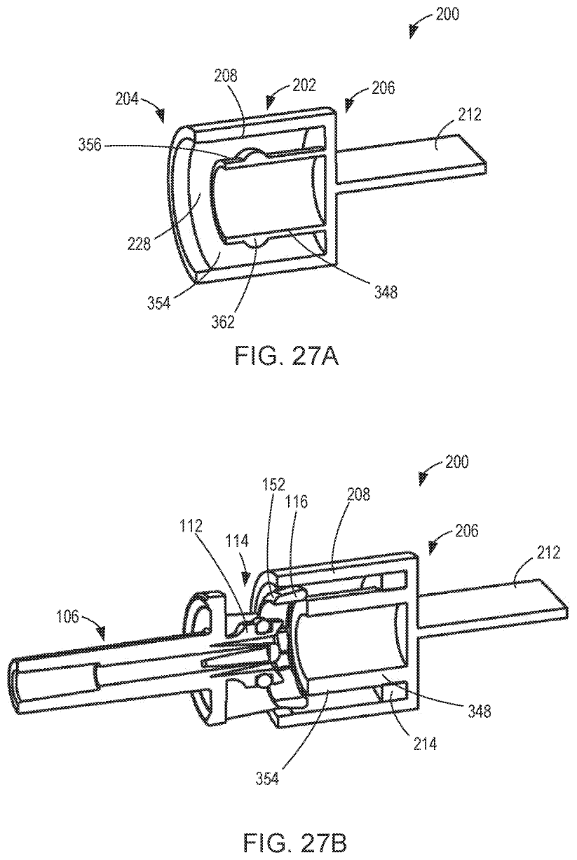

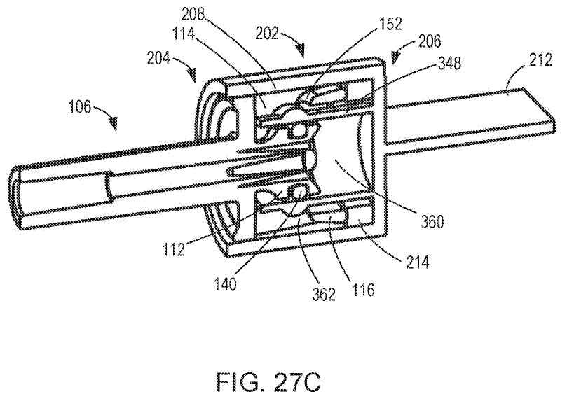

With reference to FIGS. 27A-27B, the outer surface 356 of the inner sleeve 348 may include the one or more clipping features 362 protruding radially outwardly and configured to releasably engage at least a portion of the lumen 112 of the fluid path element 106. In some embodiments, the one or more clipping features 362 may be configured as a continuous or discontinuous rib that extends around an outer circumference of the inner sleeve 348. The one or more clipping features 362 may be configured to engage with a ledge 152 on the aperture 114 of the outer skirt 116 of the fluid path element 106. The one or more clipping features 362 may retain the disinfecting cap 200 connected to the fluid path element 106 and resist the restoring force from the compressed absorbent material 214 so that the disinfecting cap 200 is retained on the fluid path element 106 for at least an appropriate amount of time for the disinfecting fluid to disinfect any microbial contaminants on the surfaces of the fluid path element 106. In certain embodiments, the one or more clipping features 362 may also provide tactile and/or audible feedback to the user that the disinfecting cap 200 has been appropriately installed on the fluid path element 106.

With reference to FIG. 27C, the inner sleeve 348 may have a width that substantially corresponds to a width of the space 354 between the lumen 112 and the outer skirt 116 of the fluid path element 106. In some embodiments, the outer surface 354 of the inner sleeve 348 may have a piloting feature, similar to the one or more retaining ribs 358 extending in a longitudinal direction of the inner sleeve 348. The piloting feature may help prevent smearing or transfer of microbial contaminants away from the end portions of the fluid path element 106 towards the inner lumen 112.

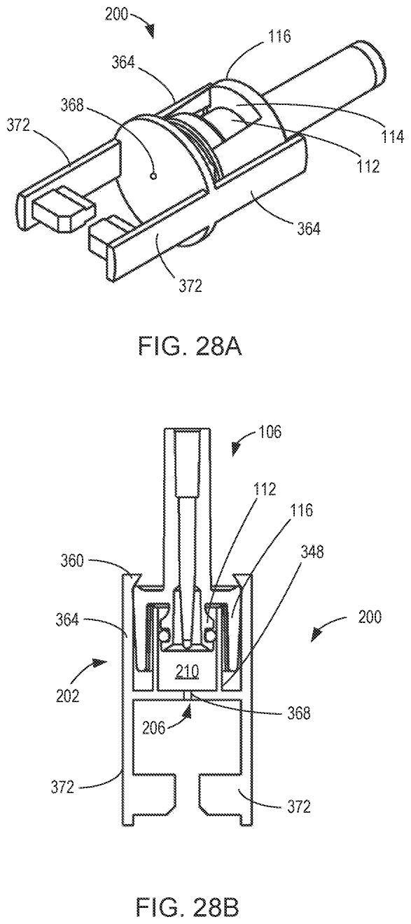

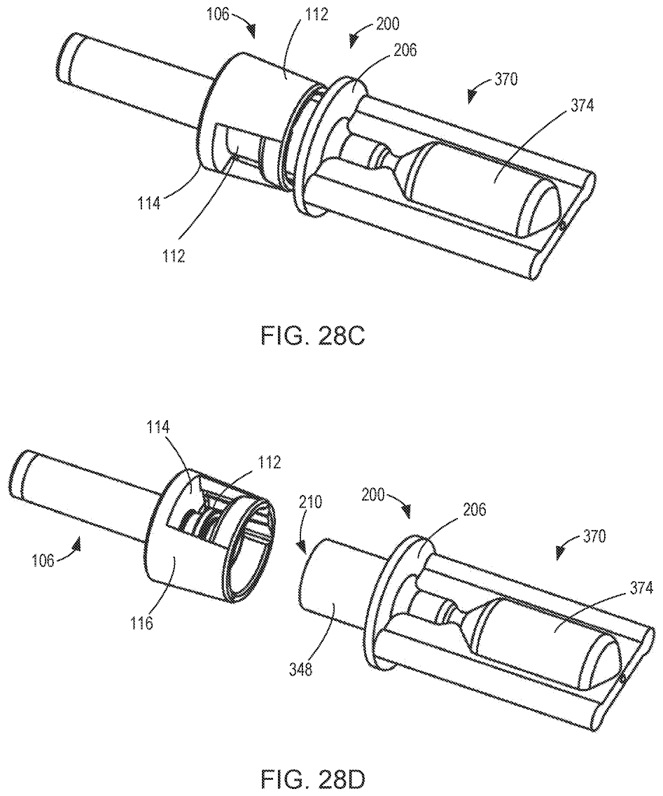

With reference to FIGS. 28A-28B, the disinfecting cap 200 may include a housing 202 having the inner sleeve 348 protruding proximally from the distal end 206. In some embodiments, the interior volume 210 may be without the compressible absorbent material, for example as according to any of the various non-limiting embodiments described herein. Two or more arms 364 extend proximally from the housing 202 and are configured to connect the housing 202 to the fluid path element 106. In some embodiments, a proximal end of each arm 364 may have a retaining clip 366 (shown in FIG. 28B) configured for engaging at least a portion of the fluid path element 106, such as the proximal end of the fluid path element 106, to retain the disinfecting cap 200 on the fluid path element 106. The lumen 112 of the fluid path element 106 may be sized to fit within the interior volume 210 of the housing 202 such that the disinfecting fluid within the interior volume 210 may contact the lumen 112.

With continued reference to FIGS. 28A-28B, the distal end 206 of the housing 202 has an opening 368 that is configured to fluidly connect to a reservoir. In some embodiments, the reservoir may be configured to contain a volume of the disinfecting fluid that may be delivered to the interior volume 210 of the housing 202 via the opening 368. The reservoir may be removably connectable to the housing 202 via one or more connecting legs 372. The reservoir may have a squeezable pouch made from a flexible material, such that, when the squeezable pouch is squeezed by the user, the disinfecting fluid exits the reservoir and enters the interior volume 210 of the housing 202 via the opening 368 to disinfect the lumen 112.

With reference to FIGS. 28C-28D, the disinfecting cap 200 may include a housing 202 having the inner sleeve 348 protruding proximally from the distal end 206. In some embodiments, the interior volume 210 may be without the compressible absorbent material, for example as according to any of the various non-limiting embodiments described herein. The lumen 112 of the fluid path element 106 may be sized to fit within the interior volume 210 of the housing 202 such that the disinfecting fluid within the interior volume 210 may contact the lumen 112. The distal end 206 of the housing 202 has an opening, similar to the opening 368 shown in FIGS. 28A-28B, which is configured to fluidly connect to a reservoir 370. In some embodiments, the reservoir 370 may be configured to contain a volume of the disinfecting fluid that may be delivered to the interior volume 210 of the housing 202 via the opening. The reservoir 370 may be removably connectable to the housing 202. In some embodiments, the reservoir 370 may be non-removably (e.g., monolithically) formed with the housing 202. The reservoir 370 may have a squeezable pouch 374 made from a flexible material. In some embodiments, a seal may be provided on the reservoir 370 to initially seal the disinfecting fluid within the reservoir 370. The seal may be configured to break when the squeezable pouch 374 is squeezed such that a sufficient fluid pressure is built in the reservoir 370. When the squeezable pouch 374 is squeezed by the user, the disinfecting fluid exits the reservoir 370 and enters the interior volume 210 of the housing 202 to disinfect the lumen 112.

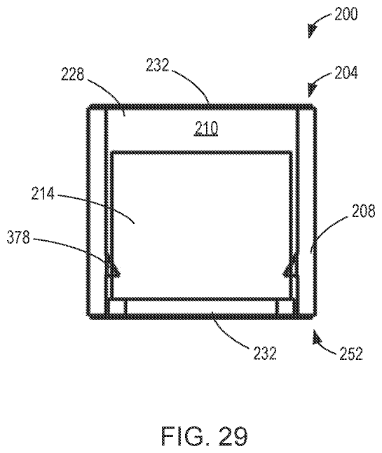

With reference to FIG. 29, the disinfecting cap 200 is shown in accordance with another embodiment. The disinfecting cap 200 has a housing 202 with an open proximal end 204 and an open distal end 252 that are each sealed with a seal 232. The seal 232 at the proximal end 204 may be removable by the user prior to connecting the disinfecting cap 200 to the fluid path element 106 (shown in FIG. 1B). The seal 232 at the open distal end 252 may be non-removably connected to the open distal end 252 to enclose one or more windows 376 that are formed in the housing 202 to allow for ready manufacture of the housing 202, such as by injection molding. The inner surface 228 of the housing 202 at the open distal end 252 may have one or more barb features 378 configured to retain the compressible absorbent material 214 within the interior volume 210 of the housing 202. In use, the user first removes the seal 232 at the open proximal end 204 of the disinfecting cap 200 and attaches the disinfecting cap 200 to the fluid path element 106 to disinfect the connector, for example by a scrubbing and/or twisting action. In certain embodiments, the entire disinfecting cap 200 may be made of foam or adsorbent material and placed in a foil or fluid proof pouch, such as a container with a removable foil seal. The user would remove the foil seal to access the foam cap and then use the disinfecting cap 200, for example a disinfecting cap made of foam but having one or more of the features described herein, to disinfect the fluid path element 200. The proximal end of the compressible absorbent material 214 may include a fluid impervious coating configured to sealably engage the lumen 112 of fluid path element 106 to prevent ingress of disinfecting fluid into the fluid path of lumen 112, as described herein.

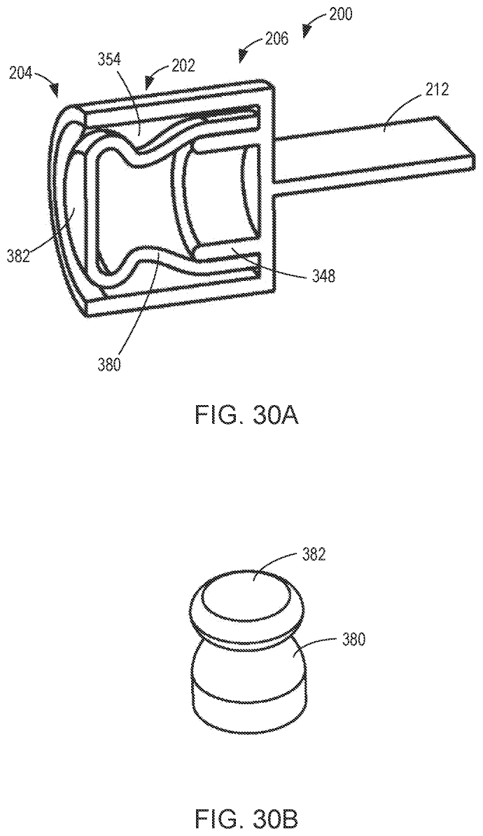

According to other embodiments, such as shown in FIGS. 30A-30B, the disinfecting cap 200 may include an elastic reservoir 380 enclosing the inner sleeve 348. The elastic reservoir 380 may be filled with a volume of the disinfecting fluid. As the disinfecting cap 200 is installed on the fluid path element 106 (shown in FIG. 1B), the elastic reservoir 380 may be compressed, thereby pressurizing the disinfecting fluid within the elastic reservoir 380. As the disinfecting fluid is compressed, the disinfecting fluid may be forced through the interface between the elastic reservoir 380 and an inner or outer surface of the inner sleeve 348. As the disinfecting fluid exits the elastic reservoir 380, the fluid fills the space 354 between the housing 202 and the inner sleeve 348 to disinfect the portion of the fluid path element 106 retained in the space 354. A proximal surface 382 of the elastic reservoir 380 may be configured to seal the lumen 112 of the fluid path element 106 (shown in FIG. 1B) to prevent ingress of the disinfecting fluid into the lumen 112.

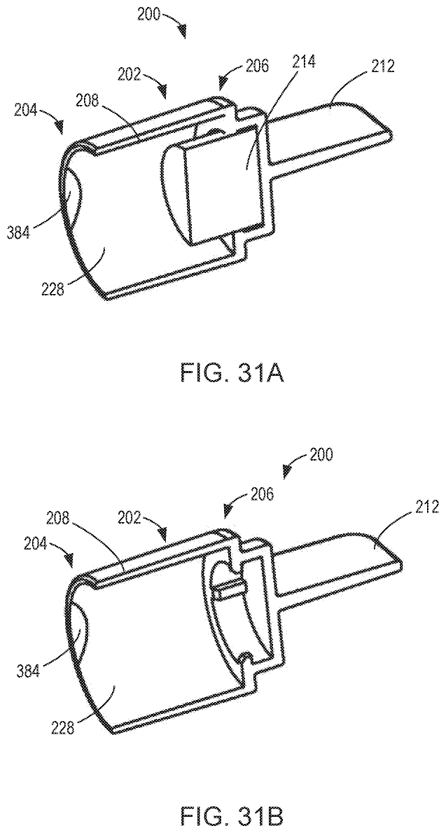

In certain embodiments, such as shown in FIGS. 31A-31B, the disinfecting cap 200 is shown in accordance with another embodiment. The inner surface 228 of the sidewall 208 of the disinfecting cap 200 may include an elliptical rim 384 and/or overall shape. The elliptical rim 384 may be defined by one or more protrusions at the proximal end 204 that define the proximal end 204 to have an elliptical shape. The elliptical rim 384 may allow an improved snap function between the projections or retaining bumps 230 (shown in FIG. 2C) on the inner surface 228 of the disinfecting cap 200 and the fluid path element 106 (shown in FIG. 1B). The elliptical rim 384 may be configured to flex into a circular shape when the disinfecting cap 200 initially contacts the fluid path element 106 to allow passage of the fluid path element 106 during connection of the fluid path element 106 with the disinfecting cap 200, and then flex back to the original elliptical shape to engage the projections or retaining bumps 230 with the fluid path element 106.

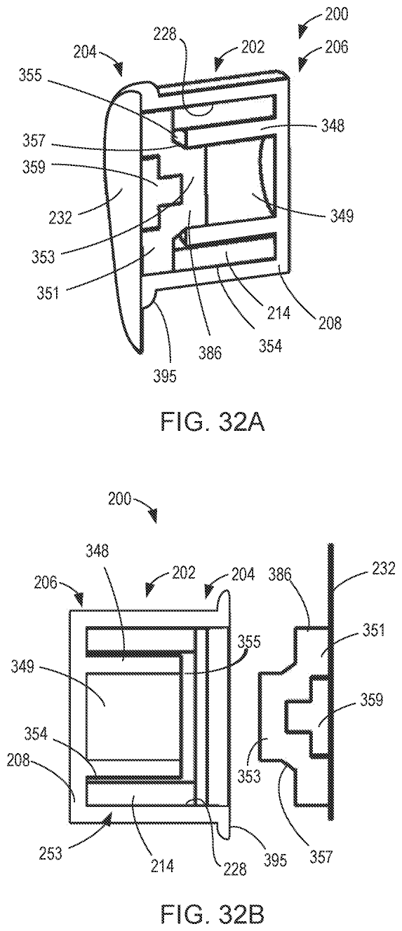

With reference to FIGS. 32A-32B, the disinfecting cap 200 is shown in accordance with another embodiment. The seal 232 of the housing 202 may have a guard 386, a portion of which is configured to be received within the interior 349 of the inner sleeve 348 when the seal 232 is connected to the open proximal end 204 of the housing 202. The guard 386 may be formed as a separate component from the seal 232 and then attached to the seal 232, such as via an adhesive. In other embodiments, the guard 386 may be monolithically formed with the seal 232, such as via molding.

A compressible absorbent material 214 is received within the space 354 between the inner surface 228 of the housing 202 and an outer surface of the inner sleeve 348. In some embodiments, the guard 386 is configured to prevent a disinfecting fluid contained within compressible absorbent material 214 from entering the interior 349 of the inner sleeve 348, such as during shipping and/or storage of the disinfecting cap 200. The guard 386 may have a stepped design with a first portion 351 having a first diameter configured to fit within the interior of the sidewall 208 and a second portion 353 having a second diameter smaller than the first diameter and protruding distally from the first portion 351. Second portion 353 may be configured to be received within the interior 349 of the inner sleeve 348. A void 359 may be provided between the first portion 351 and an inner surface of the seal 232 such that guard 386 has a substantially uniform thickness between first portion 351 and second portion 353.

A beveled portion 357 may be provided at a transition between the first portion 351 and the second portion 353. The beveled portion 357 of the guard 386 interfaces with a proximal inner edge 355 of inner sleeve 348 to form a fluid-tight seal to prevent ingress of disinfecting fluid from the compressible absorbent material 214, for example during shipping or storage. In this manner, when the seal 232 and the guard 386 are removed from the housing 202, the interior of the inner sleeve 348 will be free of the disinfecting fluid that may otherwise enter the lumen 112 of the fluid path element 106 (shown in FIG. 1B) when the disinfecting cap 200 is connected to the fluid path element 106. As the lumen 112 is inserted into the inner sleeve 348, a fluid tight seal is formed between sealing elements, for example O-rings around the outer surface of lumen 112 and the inner wall of inner sleeve 348. Compression of the compressible absorbent material 214 by the outer skirt 116 of fluid path element 106 releases the disinfecting fluid to disinfect the various exposed surfaces of fluid path element 106, as described herein. In certain embodiments, the sidewall 202 at the proximal end 204 of disinfecting cap 200 may include a widened portion 395 to increase the surface area contact and adhesion between the proximal end of sidewall 202 and the seal 232. The increased surface area contact and adhesion may prevent unintended breaking of fluid tight connection between the seal 232 and the proximal end of sidewall 202, for example during packing, shipping, unpacking, and/or storage.

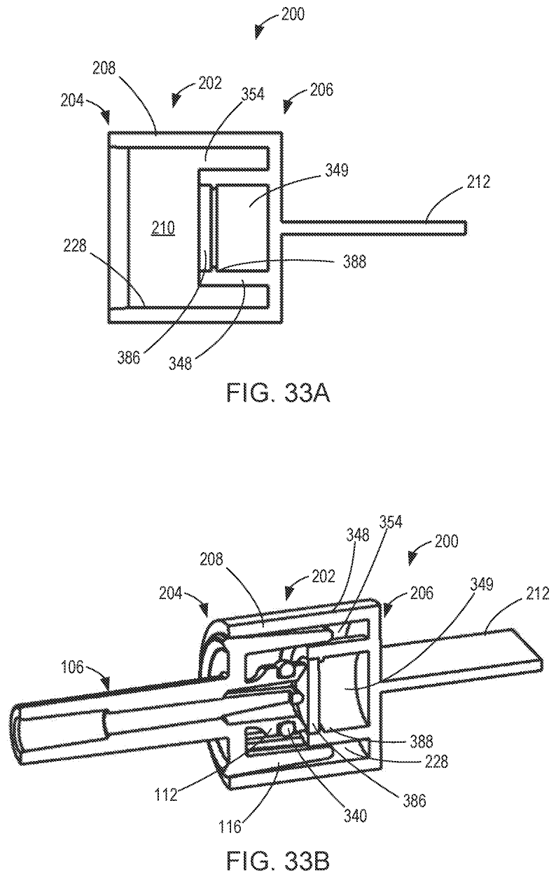

With reference to FIGS. 33A-33B, the guard 386 may be retained to the proximal end of the inner sleeve 348, for example by a notch 388. In certain embodiments, before activation of the disinfecting cap 200, the interior of the inner sleeve 348 may be filled with the disinfecting fluid and the guard 386 may seal the disinfecting fluid within an interior 349 of the inner sleeve 348. In some embodiments, compressible absorbent material, similar to the compressible absorbent material 214 described herein with reference to FIGS. 2A-2F, that is at least partially saturated with the disinfecting fluid may fill at least a portion of the interior 349 of the inner sleeve 348. The proximal end of the guard 386 may include a fluid impervious coating configured to sealably engage the lumen 112 of fluid path element 106 to prevent ingress of disinfecting fluid into the fluid path of lumen 112, as described herein.

When the fluid path element 106 is initially connected with the disinfecting cap 200 (FIG. 33B), the lumen 112 of the fluid path element 106 contacts the guard 386 such that the interior of the lumen 112 is sealed by the proximal surface of the guard 386. As the fluid path element 106 is further pushed distally into the interior volume 210 of the disinfecting cap 200, the guard 386 is displaced from the notch 388 and is moved toward the distal end 206 of the housing 202. Such movement of the guard 386 displaces the disinfecting fluid from the interior 349 of the inner sleeve 348 around the guard 386 and into the space 354 to disinfect the surfaces of the fluid path element 106.

In another embodiment, the interior of the inner sleeve 348 may be free of disinfecting fluid and the interior volume 210 may include the disinfecting fluid, such as absorbed substantially in a cylindrically oriented compressible absorbent material 214 in the space 354 between the inner sleeve 348 and the inner surface 228 of the sidewall 208, as described according to various embodiments herein. As the lumen 112 contacts the guard 386, the lumen 112 is sealed from ingress of any disinfecting fluid. Further installation of the cap compresses the compressible absorbent material 214 to release the disinfecting fluid into contact with the portions of the fluid path element 106.

With reference to FIGS. 34A, a plurality of disinfecting caps 200 may be connected to a seal strip 390. For example, any of the embodiments where the disinfecting caps 200 are described as having a seal 232, for example those described with reference to FIGS. 32A and 32B, may be provided where the seal 232 is in the form of a sealing strip 390. While FIG. 34A shows the disinfecting caps 200 without the gripping elements 212, the disinfecting caps 200 may be provided with the gripping elements 212 in some embodiments. In some embodiments, the seal strip 390 may extend across the open proximal end 204 of each disinfecting cap 200 to enclose the interior volume thereof. The seal strip 390 may be an adhesive and/or frangible seal, removably attached to and covering the open proximal end 204 of the disinfecting cap 200. The seal strip 390 allows for shipping and storage of the disinfecting cap 200 without loss of (e.g., evaporation) and or contamination of the disinfecting fluid in the interior volume of the housing 202. Each of the plurality of disinfecting caps 230 may be removed one-by-one, as needed, from the seal strip 390 without affecting the sterility or condition of the remaining disinfecting caps 200. As shown in FIG. 34A, each disinfecting cap 200 may include a widened portion 395 at the proximal end of sidewall 202 to increase the surface area contact and adhesion between the proximal end of sidewall 202 and the seal 232. The increased surface area contact and adhesion may prevent unintended breaking of fluid tight connection between the seal 232 and the proximal end of sidewall 202, for example during packing, shipping, unpacking, and/or storage. The seal strip 390 may further include a hole 397 to hang the seal strip 390, for example on the fluid injector or IV pole to allow ready access to the disinfecting caps 200 during setup of a subsequent fluid injection procedure. While each seal strip 390 may contain any number of disinfecting caps 200, it is contemplated that each seal strip 390 will contain an even number of disinfecting caps 200, as the disinfecting caps 200 are typically used in pairs for disinfection of the corresponding ends of the contrast fluid line and the saline fluid line associated with the multi-patient fluid path set 104. As shown in FIG. 34B, a plurality of seal strips 390, each having a plurality of disinfecting caps 200 connected thereto, may be packed in a box 392 for shipping and bulk storage.

While various examples of the present disclosure were provided in the foregoing description, those skilled in the art may make modifications and alterations to these examples without departing from the scope and spirit of the disclosure. Accordingly, the foregoing description is intended to be illustrative rather than restrictive. The disclosure described hereinabove is defined by the appended claims, and all changes to the disclosure that fall within the meaning and the range of equivalency of the claims are to be embraced within their scope.

1.-28. (canceled)

29. A disinfecting cap for a fluid path element having an inner lumen and an outer cylindrical wall surrounding the inner lumen, the disinfecting cap comprising:
a housing configured to receive the inner lumen and the outer cylindrical wall of the fluid path element, the housing comprising an open proximal end, a closed distal end, and a sidewall extending between the open proximal end and the closed distal end to define an interior volume;
a sleeve protruding proximally from an inner surface of the closed distal end, the sleeve defining an opening configured to receive the inner lumen of the fluid path element; and
a compressible absorbent material at least partially saturated with a disinfecting fluid, the compressible absorbent material disposed within the interior volume of the housing and surrounding at least a portion of an outer portion of the sleeve,
wherein the outer cylindrical wall of the fluid path element is configured to be received in a disinfecting space between an inner surface of the housing and the sleeve such that movement of the fluid path element toward the closed distal end compresses the compressible absorbent material and releases at least a portion of the disinfecting fluid into the disinfecting space.

30. The disinfecting cap of claim 29, wherein an inner surface of the sleeve is configured to be in sealing engagement with the inner lumen of the fluid path element.

31. The disinfecting cap of claim 29, wherein the compressible absorbent material extends substantially from the closed distal end to the proximal end of the housing.

32. The disinfecting cap of claim 29, wherein the sleeve comprises one or more longitudinal ribs protruding radially outward and configured to engage the compressible absorbent material surrounding the outer portion of the sleeve to prevent rotation of the compressible absorbent material relative to the sleeve.

33. The disinfecting cap of claim 29, wherein the compressible absorbent material has an axial slot configured to receive the outer cylindrical wall of the fluid path element.

34. The disinfecting cap of claim 29, wherein the sleeve comprises a tab configured to provide a tactile or audio feedback when the inner lumen of the fluid path element is fully inserted into the sleeve.

35. The disinfecting cap of claim 29, wherein an inner surface of the sidewall comprises one or more radially inwardly protruding projections configured to removably engage the outer cylindrical wall and retain the disinfecting cap on the fluid path element.

36. The disinfecting cap of claim 29, wherein the absorbent material is a sponge.

37. The disinfecting cap of claim 29, wherein the absorbent material is cotton.

38. The disinfecting cap of claim 29, wherein the disinfecting fluid comprises isopropyl alcohol, ethanol, a combination thereof, or an aqueous solution thereof.

39. The disinfecting cap of claim 29, further comprising a gripping flange protruding distally from the closed distal end.

40. The disinfecting cap of claim 29, further comprising a seal removably connected to the open proximal end, wherein the seal fluidly seals the open proximal end.

41. The disinfecting cap of claim 40, wherein the seal comprises a pull tab protruding radially outward relative to the sidewall of the housing, and wherein the pull tab is configured to remove the seal from the open proximal end.

42. A fluid path assembly comprising:
a fluid path element having an inner lumen and an outer cylindrical wall surrounding the inner lumen; and
a disinfecting cap configured to connect to the fluid path element, the disinfecting cap comprising:
a housing configured to receive the inner lumen and the outer cylindrical wall of the fluid path element, the housing comprising an open proximal end, a closed distal end, and a sidewall extending between the open proximal end and the closed distal end to define an interior volume;
a sleeve protruding proximally from an inner surface of the closed distal end, the sleeve defining an opening configured to receive the inner lumen of the fluid path element; and
a compressible absorbent material at least partially saturated with a disinfecting fluid, the compressible absorbent material disposed within the interior volume of the housing and surrounding at least a portion of an outer portion of the sleeve,
wherein the outer cylindrical wall of the fluid path element is configured to be received in a disinfecting space between an inner surface of the housing and the sleeve such that movement of the fluid path element toward the closed distal end compresses the compressible absorbent material and releases at least a portion of the disinfecting fluid into the disinfecting space.

43.-54. (canceled)

55. The fluid path assembly of claim 42, wherein the fluid path element is a Luer connector.

56. The fluid path assembly of claim 42, wherein the fluid path element is a multi-patient fluid path element.

57. The fluid path assembly of claim 42, wherein the fluid path element is a single-patient fluid path element.

58. The fluid path assembly of claim 42, wherein the fluid path element comprises:
a first connector element comprising a body, a first lumen, a first flexible leg, and a second flexible leg; and
a second connector element comprising a body defining an undercut, a second lumen, a channel defined in the body, and at least one sealing element positioned within the channel,
wherein the first flexible leg comprises a first flange and the second flexible leg comprises a second flange,
wherein, upon engagement of the first connector element with the second connector element, the first flange and the second flange engage with the undercut of the body of the second connector element to prevent disengagement of the first connector element and the second connector element, and
wherein the sealing element is configured to define a fluid tight seal between the second lumen of the second connector element and the first lumen of the first connector element to form a fluid path when the first connector element and the second connector element are engaged with one another.
